(19) United States
(12) Patent Application Publication
(10) Pub. No.: US 2025/0381397 A1
(43) Pub. Date: Dec. 18, 2025

(54) DEVICES AND METHODS FOR NEURAL STIMULATION

(71) Applicant: NeuraStasis, Inc., Houston, TX (US)

(72) Inventors: Kirt GILL, Milpitas, CA (US); Joseph UPCHURCH, Houston, TX (US); Andrew SUN, Houston, TX (US)

(21) Appl. No.: 19/036,863

(22) Filed: Jan. 24, 2025

Related U.S. Application Data
(63) Continuation of application No. PCT/US2023/071740, filed on Aug. 4, 2023.
(60) Provisional application No. 63/395,686, filed on Aug. 5, 2022.

(51) Int. Cl. A61N 1/36; A61N 1/04

(52) U.S. Cl. CPC A61N 1/36025; A61N 1/0456; A61N 1/0476; A61N 1/0492; A61N 1/36034; A61N 1/36036

(57) ABSTRACT

Disclosed herein are devices, systems, and methods for treating a medical condition through a coordinated stimulation of two or more target nerves using a head worn apparatus. The apparatus may include an electrode assembly for neural stimulation of a plurality of target nerves, a substrate; a set of electrodes, having: a set of frontal electrodes including a superior frontal electrode and an inferior frontal electrode, the set of frontal electrodes positioned on the substrate such that the set of frontal electrodes are configured to overlie or straddle a supraorbital trigeminal nerve branch of the subject; or a set of lateral electrodes including a concha electrode and a tragus electrode, the set of lateral electrodes positioned on the substrate such that the set of lateral electrodes are configured to overlie or straddle an auricular branch of the vagus nerve of the subject.

### CROSS-REFERENCE

This application claims the benefit of PCT/US2023/071740, filed Aug. 4, 2023, which claims the benefit of U.S. Provisional Application No. 63/395,686, filed Aug. 5, 2022, both of which are hereby incorporated by reference in its entirety herein.

### BACKGROUND

Nerve stimulation is known in the art to provide certain physiological effects on a subject. Different types of nerve stimulation include electrical nerve stimulation, chemical nerve stimulation, thermal nerve stimulation, and mechanical nerve stimulation. Electrical stimulation can be delivered as transcutaneously or percutaneously. Nerve stimulation is commonly used to alleviate pain experienced by a subject.

Cardiovascular disease is a leading cause of death globally and is responsible for about 25% of deaths in the United States. One in every 6 cardiovascular disease deaths is due to stroke, and more than 795,000 people in the United States have a stroke annually. About 87% of all strokes are ischemic strokes, in which blood flow to the brain is blocked. Stroke is a leading cause of serious long-term disability, as stroke reduces mobility in more than half of stroke survivors aged 65 and over due to brain damage resulting from the stroke. Stroke-related costs in the United States came to nearly $46 billion between 2014 and 2015. Presently, there are few commercially available treatments available to treat or mitigate an ischemic stroke prior to administration of a reperfusion therapy.

The following references may be of relevance: WO2020079218A1, WO2020185601A1, WO2020219072A1, U.S. Pat. Nos. 7,149,574B2, 7,636,597B2, 7,983,762B2, 8,055,347B2, 8,190,248B2, 8,676,330B2, 8,688,220B2, 8,702,584B2, 8,755,892B2, 8,843,210B2, 8,914,114B2, 8,918,178B2, 8,958,881B2, 9,089,691B2, 9,119,953B2, 9,174,066B2, 9,233,245B2, 9,272,157B2, 9,333,347B2, 9,358,381B2, 9,399,134B2, 9,468,763B2, 9,782,584B2, 10,058,704B2, 10,105,549B2, 10,130,809B2, 10,286,211B2, 10,293,158B2, 10,039,928B2, 10,441,780B2, 10,537,728B2, 10,537,729B2, 10,576,279B2, 10,695,568B1, 10,773,080B2, 11,260,229B2, US20070049814A1, US20150142082A1, US20170028198A1, US20170151433A1, US20180064935A1, US20180200522A1, US20190022389A1, US20190046794A1, US20190111255A1, US20190134393A1, US20,190,151604A1, US20,190,201694A1, US20,190,262229A1, US20,200,046976A1, US20,200,086108A1, US20,200,094040A1, US20,200,094055A1, US20,200,261722A1, US20,200,269046A1, US20,200,298001A1, US20,200,368527A1, US20,210,154474A1, JP5858920B2, EP1843814B1, EP2026872B1, EP3693053A1, DE102015002589B4, CN102858404B, and CA3137936A1.

### SUMMARY

The lack of commercially available treatments available to treat or mitigate an ischemic stroke prior to administration of a reperfusion therapy increases the severity of an ischemic stroke due to prolonged lack of oxygen to the region of the ischemic stroke, and the resulting brain damage. A non-invasive therapy which could increase the flow of blood or oxygen to the brain and which could be administered shortly following diagnosis and before a reperfusion therapy is administered would serve to significantly improve the treatment of ischemic stroke, improve subject outcomes, and reduce stroke-related costs. For instance, prolonged lack of oxygen to the penumbral tissue region of an ischemic stroke results in increased infarct core formation, increased brain damage. These are common negative subject outcomes because of the prolonged time between stroke diagnosis and administration of a reperfusion therapy. Similarly, the rapid reoxygenation of the penumbral tissue following administration of a reperfusion therapy may also result in a reperfusion injury, contributing to negative subject outcomes and high stroke-related costs.

It is appreciated by the inventors that nerve stimulation may be used to increase a flow of blood and oxygen to the brain or inhibit on other pathways that lead to cell death, and such an application may be useful in treating an ischemic stroke, and that non-invasive nerve stimulation may be applied quickly following diagnosis of stroke well before a reperfusion therapy can be administered. It is similarly appreciated by the inventors that nerve stimulation may be used to modulate a flow of blood and oxygen to the brain or inhibit on other pathways that lead to cell death, and such an application may be useful in treating, mitigating, or preventing a reperfusion injury resulting from a reperfusion therapy administered in conjunction with the treatment for an ischemic stroke. The devices, systems and methods described herein may be configured for treating a medical condition through a coordinated stimulation of two or more targeted nerves. The medical condition may comprise ischemic stroke, traumatic brain injury, intracranial hemorrhage (e.g., subarachnoid hemorrhage, subdural hematoma, epidural hematoma, intracerebral hemorrhage, etc.), vasospasm, cardiac arrhythmia, other conditions that involve loss of blood flow or ischemia, inflammatory diseases, conditions that can be managed through inflammatory modulation (e.g., rheumatoid arthritis, irritable bowel syndrome, sepsis, renal ischemia, trauma/hemorrhagic shock, acute lung injury, hyperinflammation, etc.), hypotension, disorders of consciousness, migraine, headache or other facial pain, other diseases that cause pain, neurological conditions (e.g., Alzheimer's Disease, Mild Cognitive Impairment, etc.), ocular conditions, infectious diseases, auditory deficits, hypoxia, or a combination thereof. The devices, systems, and methods described herein may be configured to help ease intraprocedural complications or help regulate homeostasis in the central nervous system (CNS). The devices, systems, and methods described herein may also be configured to help improve recovery from a neurological deficit or help modulate cortical or sub-cortical neuroplasticity for learning applications, for example, improving mobility in subjects having suffered brain damage following a stroke.

Aspects of the present disclosure provide electrode assemblies for neural stimulation of a plurality of target nerves in a subject. An exemplary assembly may comprise a substrate including a plurality of electrical connections and a set of electrodes. The set of electrodes may comprise one or more of (a) at set of frontal electrodes or (b) a set of lateral electrodes. The set of frontal electrodes may include a superior frontal electrode and an inferior frontal electrode. The set of frontal electrodes may be positioned on the substrate such that the set of frontal electrodes are configured to contact the subject's skin and to one or more of overlie or straddle a supraorbital trigeminal nerve branch of the subject. The set of lateral electrodes may include a concha electrode and a tragus electrode. The set of lateral electrodes may be positioned on the substrate such that the set of lateral electrodes are configured to contact the subject's skin and to one or more of overlie or straddle an auricular branch of the vagus nerve of the subject. The substrate may include a fixation promoter configured to promote contact with each electrode and the subject's skin. Each electrode may be in communication with at least one independent electrical connection of the plurality of electrical connections. The exemplary electrode assembly may further comprise a second set of frontal electrodes including a second superior frontal electrode and a second inferior frontal electrode. The second set of frontal electrodes may be positioned on the substrate such that the second set of frontal electrodes are configured to contact the subject's skin and to one or more of overlie or straddle a second supraorbital trigeminal nerve branch of the subject. The exemplary electrode assembly may further comprise a second set of lateral electrodes including a second concha electrode and a second tragus electrode. The second set of lateral electrodes may be positioned on the substrate such that the second set of lateral electrodes are configured to contact the subject's skin and to one or more of overlie or straddle a second auricular branch of the vagus nerve of the subject. The exemplary electrode assembly may further comprise an adhesive layer surrounding each electrode configured to adhere the electrodes to the skin of the subject. In some embodiments, the sets of frontal electrodes may be symmetrically oriented from a midline of the electrode assembly. In some embodiments, at least one of the electrodes comprises a hydrogel configured to enhance current transmission between a pulse generator and a respective target nerve. In some embodiments, a pulse generator may be configured to transmit current from the superior frontal electrode to the inferior frontal electrode. In some embodiments, the inferior frontal electrode is shaped to ensure coverage of a nerve bundle above the supraorbital foramen or notch. In some embodiments, the superior frontal electrode has a triangular shape configured to generate an electric field in an orientation of the supraorbital trigeminal nerve branch. The triangular shape of the superior frontal electrode may be configured to reduce a current density reducing nerve fibers that will be recruited on a secondary phase of a pulse of a pulse generator. The triangular shape of the superior frontal electrode may be configured to generate an electric field in an orientation of the targeted nerve branch. In some embodiments, the concha electrode is configured to fit in a respective cymba concha of the subject and below the anti-helix of the subject and above the helicis crus of the subject. In some embodiments, the substrate between the concha electrode and the tragus electrode has an arc shape configured to follow a contour of the helicis crus of the subject and to allow additional surface area for adhesion within the cavum concha of the subject. In some embodiments, the substrate between the concha electrode and the tragus electrode has an arc shape configured to follow a contour of the antihelix of the subject. The exemplary electrode assembly may further comprise a mechanical tensioner substantially along at least a portion of a length of the electrode assembly configured to apply a compressive pressure to cause the electrode portions to maintain contact with the skin of a user. The mechanical tensioner may include a rigid layer within the electrode assembly configured to apply a compressive pressure. In some embodiments, the fixation promoter is at least one of an elastic headband or frame, stretchable fabric, adhesive tape, or some combination thereof. In some embodiments, the substrate includes a frontal section where the set of frontal electrodes is positioned and a lateral section where the set of lateral electrodes is positioned. The substrate may be unitary and continuous between the frontal and lateral sections. In some embodiments, the substrate includes a frontal section where the set of frontal electrodes is positioned and a lateral section where the set of lateral electrodes is positioned, and wherein the substrate is detachable from the frontal and lateral sections. In some embodiments, either or both of the lateral electrodes may be detachable from the set of frontal electrodes such that either lateral electrode or frontal electrodes can be included or omitted in the array. The detachment may be made via a connector on the substrate located between the frontal electrodes either or both lateral electrodes. In some embodiments, only one of the lateral electrodes may be utilized. In some embodiments, the electrode assembly comprises both (a) the set of frontal electrodes and (b) the set of lateral electrodes. In some embodiments, the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject. In some embodiments, the triangular shape of the superior frontal electrode reduces a current density applied to nerve fibers, reducing the nerve fibers that are recruited on a secondary phase of a pulse of a pulse generator. In some embodiments, the superior frontal electrode has a triangular shape and reduces the area of an electric field produced by the electrode. In some embodiments, the triangular shape reduces the area of an electric field produced by the electrode as compared to a semicircular shaped electrode. In some embodiments, the triangular shape increases a stimulation effect configured to be applied to the subject at a same stimulation intensity. In some embodiments, the stimulation effect configured to be applied to the subject at the same stimulation intensity is increased relative to semicircular shaped electrode. In some embodiments, the triangular shape reduces off-target nerve activations when an electrical field is generated between the superior frontal electrode and the inferior frontal electrode. In some embodiments, the set of lateral electrodes generates an electrical field in an orientation of an auricular branch of the vagus nerve. In some embodiments, the orientation of the electrical field is parallel to one or more nerve fibers in the auricular branch of the vagus nerve. In some embodiments, the substrate comprises a frontal section comprising the set of frontal electrodes a lateral section comprising to the set of lateral electrodes, wherein the lateral section is removable from the frontal section. In some embodiments, the substrate is unitary and continuous between the frontal section and the lateral section. In some embodiments, the frontal section, is detachable from the lateral section. In some embodiments, the electrode assembly comprises the set of frontal electrodes and the set of lateral electrodes. In some embodiments, the set of lateral electrodes are removable from the device. In some embodiments, each electrode in the first set of electrodes is in independent communication with at least one of the plurality of electrical connections the electrical connection. In some embodiments, a position of the concha electrode and the tragus electrode minimize off-target nerve activations when an electrical field is generated between the concha electrode and the tragus electrode and applied to the subject. In some embodiments, the electrode assembly is comprised in a head-worn apparatus for neural stimulation of target nerves in a subject, wherein the target nerve are the auricular branch of the vagus nerve and the supraorbital trigeminal nerve branch.

Aspects of the present disclosure provide methods of treating or augmenting recovery from a medical condition of a subject. An exemplary method may comprise the step of contacting the exemplary electrode assembly with the skin of the subject so that one or more of (a) the set of frontal electrodes one or more of overlie or straddle the supraorbital trigeminal nerve branch of the subject, or (b) the set of lateral electrodes one or more of overlie or straddle the auricular branch of the vagus nerve of the subject. The exemplary method may further comprise the step of transcutaneously delivering stimulation energy through one or more of the set of frontal electrodes or the set of lateral electrodes to treat the medical condition. The medical condition may be one or more of ischemic stroke, cerebral brain damage due to ischemic stroke, hemorrhagic stroke, cerebral brain damage due to hemorrhagic stroke, a reperfusion injury, traumatic brain injury, subarachnoid hemorrhage, a migraine, a headache, a form of dementia or cognitive impairment, a hematoma, a hemorrhage, a subarachnoid hemorrhage, inflammation, hypertension, hypotension, brain damage resulting from brain surgery, brain damage resulting from a brain resection, multiple sclerosis or lesions therefrom, cerebral palsy, or combinations thereof. In some embodiments, treating or augmenting recovery from a medical condition comprises stroke rehabilitation. In some embodiments, the method includes contacting the electrode assembly with the skin of the subject so that both (a) and (b) occur. In some embodiments, transcutaneously delivering stimulation energy comprises increasing a blood flow to the brain. In some embodiments, transcutaneously delivering stimulation energy comprises increasing a mean blood flow velocity to the brain, an increase in end diastolic velocity, a decrease in pulsatility index, or combinations thereof. In some embodiments, the superior frontal electrode has a triangular shape. In some embodiments, transcutaneously delivering stimulation energy comprises generating an electric field in an orientation of the supraorbital trigeminal nerve branch. In some embodiments, the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject. In some embodiments, transcutaneously delivering stimulation energy comprises reducing a current density applied to nerve fibers, and reducing the nerve fibers that are recruited upon generating a secondary phase of a pulse with a pulse generator. In some embodiments, transcutaneously delivering stimulation energy comprises reducing the area of an electric field produced by the superior frontal electrode relative to a semicircular shaped electrode. In some embodiments, transcutaneously delivering stimulation energy comprises increasing a stimulation effect to the subject at the same stimulation intensity relative to a stimulation effect resulting from a semicircular shaped electrode. In some embodiments, transcutaneously delivering stimulation energy comprises reducing off-target nerve activations when an electrical field is generated between the superior frontal electrode and the inferior frontal electrode and applied to the subject. In some embodiments, transcutaneously delivering stimulation energy comprises generating an electrical field in an orientation of an auricular branch of the vagus nerve with the set of lateral electrodes. In some embodiments, the orientation of the electrical field is parallel to one or more nerve fibers in the auricular branch of the vagus nerve. In some embodiments, transcutaneously delivering stimulation energy comprises minimizing off-target nerve activations when an electrical field is generated between the concha electrode and the tragus electrode and applied to the subject. In some embodiments, transcutaneously delivering stimulation energy comprises reducing off-target nerve activations when an electrical field is generated between the superior frontal electrode and the inferior frontal electrode. In some embodiments, the method includes attaching the electrode assembly to a head of the subject, wherein the electrode assembly is comprised in a head-worn apparatus for neural stimulation of target nerves in a subject, wherein the target nerve are the auricular branch of the vagus nerve and the supraorbital trigeminal nerve branch.

Aspects of the present disclosure provide head worn apparatuses for neural stimulation of a plurality of target nerves in a subject. An exemplary head worn apparatus may comprise one or more of (a) a forehead section or (b) a lateral section. The forehead section may have a shape and area configured to overlap with a supraorbital trigeminal nerve branch of the subject when worn by the subject, and the forehead section may include: a set of frontal stimulation elements positioned such that the set of frontal stimulation elements are configured to one or more of overlie or straddle the supraorbital trigeminal nerve branch when worn by the subject, and a frontal layer configured to maintain contact between the set of frontal stimulation elements and the subject's skin at the forehead when the apparatus is worn. The lateral section may include: a set of lateral stimulation elements including a concha stimulation element and a tragus stimulation element positioned such that the set of lateral stimulation elements are configured to one or more of overlie or straddle the auricular branch of the vagus nerve when the lateral section is worn by the subject, and a lateral layer configured to maintain contact between the set of lateral stimulation elements and the subject's skin when the apparatus is worn. The exemplary heard worn apparatus may further comprise (c) a connector assembly extending from at least one of the forehead section or the lateral section and including a plurality of connections in communication with at least one of the set of frontal stimulation elements or the set of lateral stimulation elements. The exemplary head worn apparatus may further comprise a pulse generator connected to the connector assembly. The exemplary head worn apparatus may further comprising a temple section. The temple section may extend laterally from the forehead section at an angle transverse to a major axis of the forehead section. The lateral section may be connected to the temple section. The connector assembly may extend from temple section. When the apparatus is worn, the temple section may leave open an ultrasonic window exposing skin of the subject to allow for transcranial Doppler ultrasound assessment of cerebral arteries of the subject without removal of the apparatus. The temple section may be configured to leave skin of the subject over a temporal bone of the subject exposed when the apparatus is worn. In some embodiments, the lateral section between the concha electrode and the tragus electrode has an arc shape configured to follow a contour of a helicis crus of the subject and to allow additional surface area for adhesion within a cavum concha of the subject. In some embodiments, an area of the forehead section is configured to provide maximal coverage over the forehead of the subject. That is, all exposed skin may be covered with the adhesive to obtain the highest surface area for bonding. In some embodiments, when the notch is positioned centered between the subject's eyebrows, a lower edge of the forehead section begins to wrap to below the upper orbit and above an eyelid of the subject. In some embodiments, the exemplary head worn apparatus further comprises a mechanical tensioner substantially configured to apply a compressive pressure to cause at least one of the set of frontal stimulation elements or the set of lateral stimulation elements to maintain contact with the skin of the subject. The mechanical tensioner may extend from at least one of the forehead section or the temple section. The mechanical tensioner may be at least one of a strap or a curved material under tension. In some embodiments, the fixation promoter is a stretchable fabric assembly with a hook fastener (e.g., Velcro®) on an inner face of approximately 2.25″ in length and loop fastener (e.g., Velcro®) on an outer face of a longer length of approximately 6″ in length. The fabric assembly wraps circumferentially around the subject's head, cover the frontal electrode locations, where the difference in the lengths of the hook and loop fasteners allows for tensioning of the fabric to fit different head circumferences and while maintaining electrode contact with the skin. In some embodiments, the connector assembly comprises a set of connectors coupled to one or more of the set of lateral stimulation elements or the set of frontal stimulation elements. The connector assembly may further comprise a base layer coupled to the set of connectors. The connectors may be flexible.

In some embodiments, the base layer is unitary and continuous between one or more of the forehead, temple, or lateral sections of the apparatus. In some embodiments, the exemplary head worn apparatus further comprises a notch in the bottom middle configured to facilitate positioning the forehead section centered from the midline of the subject's forehead. In some embodiments, the set of frontal stimulation elements comprise a set of frontal electrodes. In some embodiments, the set of lateral stimulation elements comprise a set of lateral electrodes. In some embodiments, the exemplary head worn apparatus comprises both (a) the forehead section or (b) the lateral section. In some embodiments, the frontal layer is adhesive. In some embodiments, the lateral layer is adhesive. In some embodiments, multiple releaser liners are used on and around the hydrogel electrodes such that removal initially only exposes the hydrogel and removal of a second release liner exposes the electrode adhesive. In some embodiments, the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject. In some embodiments, the triangular shape of the superior frontal electrode reduces a current density applied to nerve fibers, reducing the nerve fibers that are recruited on a secondary phase of a pulse of a pulse generator. In some embodiments, the superior frontal electrode has a triangular shape and reduces the area of an electric field produced by the electrode. In some embodiments, the triangular shape reduces the area of an electric field produced by the electrode as compared to a semicircular shaped electrode. In some embodiments, the triangular shape increases a stimulation effect configured to be applied to the subject at a same stimulation intensity. In some embodiments, the stimulation effect configured to be applied to the subject at the same stimulation intensity is increased relative to semicircular shaped electrode. In some embodiments, the triangular shape reduces off-target nerve activations when an electrical field is generated between the superior frontal electrode and the inferior frontal electrode. In some embodiments, the set of lateral electrodes generates an electrical field in an orientation of an auricular branch of the vagus nerve. In some embodiments, the orientation of the electrical field is parallel to one or more nerve fibers in the auricular branch of the vagus nerve. In some embodiments, the substrate comprises a frontal section comprising the set of frontal electrodes a lateral section comprising to the set of lateral electrodes, wherein the lateral section is removable from the frontal section. In some embodiments, the substrate is unitary and continuous between the frontal section and the lateral section. In some embodiments, the frontal section, is detachable from the lateral section. In some embodiments, the electrode assembly comprises the set of frontal electrodes and the set of lateral electrodes. In some embodiments, the set of lateral electrodes are removable from the device. In some embodiments, each electrode in the first set of electrodes is in independent communication with at least one of the plurality of electrical connections the electrical connection. In some embodiments, a position of the concha electrode and the tragus electrode minimize off-target nerve activations when an electrical field is generated between the concha electrode and the tragus electrode and applied to the subject. In some embodiments, the electrode assembly is comprised in a head-worn apparatus for neural stimulation of target nerves in a subject, wherein the target nerve are the auricular branch of the vagus nerve and the supraorbital trigeminal nerve branch

Aspects of the present disclosure provide methods of treating a medical condition of a subject. An exemplary method may comprise a step of contacting the exemplary head worn apparatus with the skin of the subject so that one or more of (a) the set of frontal stimulation elements one or more of overlie or straddle the supraorbital trigeminal nerve branch of the subject, or (b) the set of lateral stimulation elements one or more of overlie or straddle the auricular branch of the vagus nerve of the subject. The exemplary method may further comprise a step of transcutaneously delivering stimulation energy through one or more of the set of frontal stimulation elements or the set of lateral stimulation elements to treat the medical condition. The medical condition may be one or more of ischemic stroke, cerebral brain damage due to ischemic stroke, hemorrhagic stroke, cerebral brain damage due to hemorrhagic stroke, a reperfusion injury, traumatic brain injury, subarachnoid hemorrhage, a headache, a migraine, a form of dementia or cognitive impairment, a hematoma, a hemorrhage, a subarachnoid hemorrhage, inflammation, hypertension, hypotension, brain damage resulting from brain surgery, brain damage resulting from a brain resection, multiple sclerosis or lesions therefrom, cerebral palsy, or combinations thereof. The stimulation energy may comprise one or more of electrical, mechanical, vibratory, acoustic, optical, or thermal energy. In some embodiments, the method includes contacting the electrode assembly with the skin of the subject so that both (a) and (b) occur. In some embodiments, transcutaneously delivering stimulation energy comprises increasing a blood flow to the brain. In some embodiments, transcutaneously delivering stimulation energy comprises increasing a mean blood flow velocity to the brain, an increase in end diastolic velocity, a decrease in pulsatility index, or combinations thereof. In some embodiments, the superior frontal electrode has a triangular shape. In some embodiments, transcutaneously delivering stimulation energy comprises generating an electric field in an orientation of the supraorbital trigeminal nerve branch. In some embodiments, the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject. In some embodiments, transcutaneously delivering stimulation energy comprises reducing a current density applied to nerve fibers, and reducing the nerve fibers that are recruited upon generating a secondary phase of a pulse with a pulse generator. In some embodiments, transcutaneously delivering stimulation energy comprises reducing the area of an electric field produced by the superior frontal electrode relative to a semicircular shaped electrode. In some embodiments, transcutaneously delivering stimulation energy comprises increasing a stimulation effect to the subject at the same stimulation intensity relative to a stimulation effect resulting from a semicircular shaped electrode. In some embodiments, transcutaneously delivering stimulation energy comprises reducing off-target nerve activations when an electrical field is generated between the superior frontal electrode and the inferior frontal electrode and applied to the subject. In some embodiments, transcutaneously delivering stimulation energy comprises generating an electrical field in an orientation of an auricular branch of the vagus nerve with the set of lateral electrodes. In some embodiments, the orientation of the electrical field is parallel to one or more nerve fibers in the auricular branch of the vagus nerve. In some embodiments, transcutaneously delivering stimulation energy comprises minimizing off-target nerve activations when an electrical field is generated between the concha electrode and the tragus electrode and applied to the subject. In some embodiments, transcutaneously delivering stimulation energy comprises reducing off-target nerve activations when an electrical field is generated between the superior frontal electrode and the inferior frontal electrode. In some embodiments, the method includes attaching the electrode assembly to a head of the subject, wherein the electrode assembly is comprised in a head-worn apparatus for neural stimulation of target nerves in a subject, wherein the target nerve are the auricular branch of the vagus nerve and the supraorbital trigeminal nerve branch.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

PCT application no. PCT/US22/24988 is herein incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 provides a top view of an exemplary stimulation device showing its circuitry, in accordance with embodiments disclosed herein;

FIG. 2A provides a front-right perspective view of a subject wearing an exemplary stimulation device, in accordance with embodiments disclosed herein;

FIG. 2B provides another front-right perspective view of a subject wearing an exemplary stimulation device, in accordance with embodiments disclosed herein;

FIG. 3 provides a front view of a subject wearing an exemplary stimulation device, in accordance with embodiments disclosed herein, with underlying nerves shown;

FIG. 4 provides a side view of a subject left ear of a subject wearing an exemplary stimulation device, in accordance with embodiments disclosed herein;

FIG. 5 provides a rear perspective view showing an exemplary stimulation device, in accordance with embodiments disclosed herein;

FIG. 6 provides a graph comparing mean flow velocity and pulsatility index of an exemplary stimulation device according to embodiments disclosed herein to those of an existing device available in the market;

FIG. 7A provides a graph comparing mean flow velocity of an exemplary stimulation device according to embodiments disclosed herein to that of an existing device available in the market;

FIG. 7B provides a graph comparing the pulsatility index of an exemplary stimulation device according to embodiments disclosed herein to those of an existing device available in the market;

FIG. 8A provides a correlation graph of the effect on mean flow velocity of an exemplary stimulation device according to embodiments disclosed herein from in a study of 10 participants with risk factors for stroke;

FIG. 8B provides a correlation graph of the effect on end diastolic velocity of an exemplary stimulation device according to embodiments disclosed herein from in a study of 10 participants with risk factors for stroke;

FIG. 9 provides an isometric, exploded perspective view of an exemplary stimulation device showing detachable lateral electrodes from the substrate of the frontal electrodes and with a stretchable fabric fixation, in accordance with embodiments disclosed herein; and

FIG. 10 provides an isometric, exploded perspective view of an exemplary stimulation device showing the layering of release liners over one of the lateral electrodes, in accordance with embodiments disclosed herein.

### DETAILED DESCRIPTION

Provided herein are devices, systems, and methods for treating a medical condition through a coordinated stimulation of two or more target nerves. The stimulation may comprise electrical stimulation delivered to a subject. The coordinated stimulation may comprise the two or more target nerves being stimulated in parallel, in series, a combination thereof, or any coordinated temporal sequence. The stimulation delivery may be contingent on an electrode placement confirmation via one or more electrode monitors. The delivered electrical stimulation may comprise one or more stimulation parameters configured to be adjusted based on feedback from the subject. The feedback may comprise automatic detection of a subject physiological parameter or other subject response (e.g., muscle spasm or contraction detection). The feedback may comprise subject provided input, such as verbally or visually (e.g., facial expression, gestures). The current for electrical stimulation may be generated by a pulse generator. The device may comprise two or more sets of electrodes for electrical stimulation. The electrodes may be configured to increase effectiveness of electrical stimulation. Changes in electrical fields due to improved electrode configuration can decrease morbidity and tissue deoxygenation and degradation due to stroke. The device can be adhered to the skin. The device may comprise a rigid external frame. In some embodiments, the external frame may not adhere to the skin. In some cases, the external frame can comprise the stimulation electrodes.

Disclosed herein are systems, devices, and methods configured for combined transcutaneous or minimally invasive stimulation of the auricular branch of the vagus nerve and branches of the trigeminal nerve to slow cerebral brain damage by increasing cerebral blood flow, down-regulating the immune response, modulating nitric oxide expression, and/or interrupting ischemic depolarization, in the setting of ischemic stroke. A physiological monitoring system provided herein comprising an electroencephalogram (EEG), near-infrared spectroscopy, or other technology may be provided to detect cerebral blood flow, or any other key vital to monitor and provide safe stimulation ranges.

### Medical Condition

The devices, systems and methods described herein may be configured for treating a medical condition through a coordinated stimulation of two or more targeted nerves. The medical condition may comprise ischemic stroke, traumatic brain injury, intracranial hemorrhage (e.g., subarachnoid hemorrhage, subdural hematoma, epidural hematoma, intracerebral hemorrhage, etc.), vasospasm, cardiac arrhythmia, other conditions that involve loss of blood flow or ischemia, inflammatory diseases, conditions that can be managed through inflammatory modulation (e.g., rheumatoid arthritis, irritable bowel syndrome, sepsis, renal ischemia, trauma/hemorrhagic shock, acute lung injury, hyperinflammation, etc.), hypotension, disorders of consciousness, migraine, headache or other facial pain, other diseases that cause pain, neurological conditions (e.g., Alzheimer's Disease, Mild Cognitive Impairment, etc.), ocular conditions, infectious diseases, auditory deficits, hypoxia, or a combination thereof. The devices, systems, and methods described herein may be configured to help ease intraprocedural complications or help regulate homeostasis in the central nervous system (CNS).

### Target Nerves

“Target nerve,” as used herein, can refer to any of the following: 1) a single target nerve corresponding to one or more locations on a single branch of the target nerve; 2) a single target nerve corresponding to one or more locations on two different branches of the target nerve. For example, the supraorbital nerve comprises nerve branches located on both sides of a subject head, wherein systems and methods described herein may be configured to stimulate one or both branches.

The target nerve(s) may comprise a vagus nerve, a trigeminal nerve, a facial nerve, an auricular nerve, or a combination thereof. The target nerve(s) may comprise neural ganglion (ganglia) or nucleus (nuclei) comprising sphenopalatine ganglion, geniculate ganglion, otic ganglion, ciliary ganglion, nucleus ambiguous, spinal trigeminal nucleus, solitary nucleus, trigeminal ganglion, or some combination thereof. The vagus nerve may comprise an auricular branch, a pharyngeal nerve, a superior laryngeal nerve, superior cervical cardiac branches of the vagus nerve, or a combination thereof. The trigeminal nerve may comprise an auriculotemporal branch, a supratrochlear branch, a supraorbital branch, a maxillary branch, an ophthalmic branch, infraorbital branch, or a combination thereof. The facial nerve may comprise the greater petrosal nerve, nerve to the stapedius, chorda tympani, posterior auricular nerve, temporal branch, zygomatic branch, buccal branch, marginal mandibular branch, cervical branch, or a combination thereof. An auricular nerve may comprise the anterior branch of the greater auricular nerve, the posterior branch of the greater auricular nerve, a cutaneous branch, the nerve origin at the cervical plexus, or any combination thereof. In some embodiments, the target nerve(s), nucleus (nuclei), ganglion (ganglia), or some combination thereof comprises a sympathetic nerve, a parasympathetic nerve, a sensory nerve, a motor nerve, or a combination thereof. The target nerve(s) may comprise of sensory nerve fiber(s) Aα, Aβ, Aβ, C, or a combination thereof. The target nerve fiber(s) may have diameters range from 0.2 to 25 μm.

Systems and methods described herein may be configured to target a nucleus (e.g., nucleus tractus solitarius (NTS) sensory nuclei in the brainstem, spinal trigeminal nucleus, the superior salivatory nucleus, or the rostral ventromedial medulla). Targeting a nucleus may comprise 1) appropriate charge density at a required depth, 2) minimally invasive approach, or 3) indirect activation through downstream stimulation (via peripheral nerves).

### Nerve Stimulation

As described herein, systems, devices, and methods may be configured to stimulate two or more target nerves to treat a medical condition. The target nerves may be stimulated via electrical stimulation. Electrical stimulation may comprise delivering an electric current (e.g., electric impulses) to a subject to stimulate the respective target nerves. The electric delivery may be transcutaneous, percutaneous, and/or subcutaneous.

### Pulse Generator

The purpose of the stimulation device can include providing electrical stimulation to target nerves transcutaneously. The current necessary for generating this electrical stimulation may be delivered from a pulse generator. The stimulation device may be connected to the pulse generator at a portion of the flexible circuit designed to interface with a connector of the pulse generator. The portion of the flexible circuit may be reinforced to prevent damage during insertion, removal, and/or reinsertion. The flexible circuit portion that interfaces with the connector may be positioned as an elongated tail of the stimulation device that may not comprise adhesive or contact the subject's skin. The pulse generator may be integrated into the stimulation device and/or adhered to the external side of an adhesive layer. The pulse generator may be contained within or on an external head frame attached to the adhesive layer.

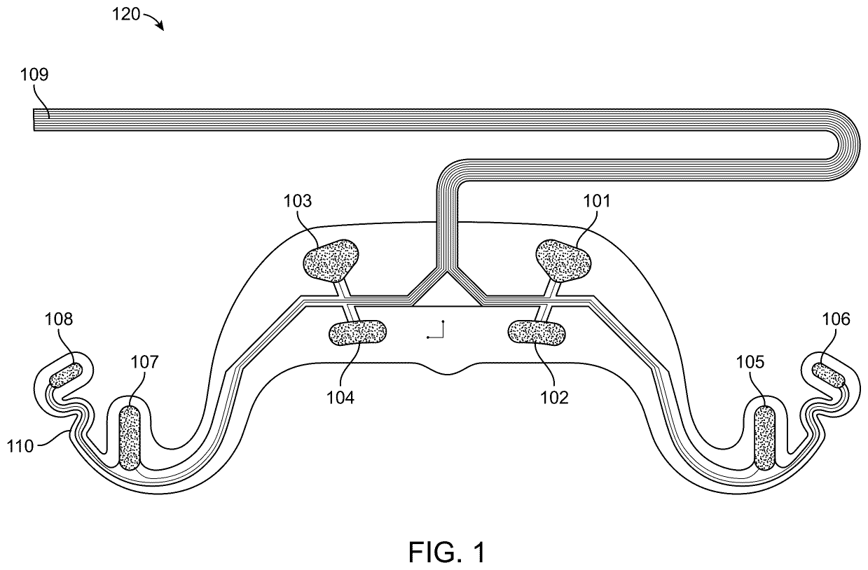

FIG. 1 provides a top view of a flat manufactured state of an exemplary stimulation device. FIG. 1 comprises a right supraorbital nerve location upper triangular electrode 101, a left supraorbital nerve location upper triangular electrode 103, a right supraorbital nerve location lower electrode 102, a left supraorbital nerve location lower electrode 104, a right vagus nerve location larger elliptical electrode 105, a left vagus nerve location larger elliptical electrode 107, a right vagus nerve location smaller elliptical electrode 106, a left vagus nerve location smaller elliptical electrode 108, and a reinforced interface for an external pulse generator connection 109. The stimulation device may be a biphasic stimulation device comprising a first phase of a pulse and a secondary phase of a pulse. The stimulation device can comprise one or more of a forehead section comprising the supraorbital electrodes, a lateral section comprising the vagus nerve electrodes, and a temple section extending laterally from the forehead section and connecting the forehead and lateral sections. The temple section can connect the supraorbital and vagus nerve electrodes in one device. The temple section can make application of the device easier and/or quicker, as the stimulation device can wrap around the side of the face.

### Stimulation Device Construction

One function of the stimulation device may be to maintain the proper positioning of the electrodes within each electrode set and for each electrode set relative to each other. Another device function may be to maintain adherence of the electrode to the skin overlying the target nerve branches. Another function of the device may be to provide a conduit between the electrodes and a pulse generator for electrical current. Another function of the device may be to distribute electrical current from the pulse generator through the skin to stimulate the target nerves. The device may be constructed such that the electrodes are in contact with the skin, a flexible circuit overlies the electrodes, and adhesive tape overlies the flexible circuit.

The shape of the device can provide sufficient area for an adhesive layer to bond to the skin in areas not overlying the electrodes or the flexible circuit. The shape of the device may minimize application of the adhesive layer to areas of hair of the user, such as the eyebrows, scalp, and sideburns. In some embodiments, the shape of the device may not cover the temporal bone and leaves open ultrasonic windows to allow for transcranial Doppler ultrasound assessment of the cerebral arteries without removal of the device. In some embodiments, the shape of the device does may overlie the eyes of the user or prevent assessment of facial droop of the user. In some embodiments, the device materials may not be rigid, so the device can be shaped to accommodate the facial anatomy of the user and allow for slight adjustments to ensure the electrodes overlie the intended nerve targets.

The stimulation device may be used in conjunction with a rigid external frame. The external frame may overlie the electrodes of the device and applies a force to the device in the direction of the skin. This force can have the effect of minimizing the thickness of skin and underlying dermal tissue, fat, and/or muscle separating the electrode and the target nerve. The frame may be constructed with a rigid front that overlies the positions of the electrodes and a flexible head strap that allows attachment of the device to the head of the subject. An additional head strap may be positioned superiorly over the head of the subject between the ears to prevent the external frame from moving inferiorly. The external frame may be injection molded and constructed from a suitable plastic including, but not limited to, polypropylene. The external frame may have adjustable components that allow repositioning of the portions in contact and applying force to the electrodes, for example to allow for adjustment between the electrodes on the forehead and the electrodes in the bilateral ears. A force applied to the electrodes may be generated as a result of the material properties of the headframe and the geometry that causes an interference fit between the head frame and the electrodes overlying the subject's skin. A force may be generated between the adjustable components and the rest of the headframe using a spring, such as a torsion spring. The spring may have a spiral-shaped configuration. The spiral may be a shape with windings about a central axis. The windings may gradually widen or tighten along the length. The spiral may be continuous. The spring may have a conical-shaped deployed configuration including, but not limited to, tubular, conical, frustoconical, or helical shapes.

Additional detail on fabric construction to reflect current design. The fixation device may be instead constructed from a stretchable fabric, such as polyester spandex. There may be two layers of fabric with the electrode substrate located between the two fabric layers. Cutouts in the interior patient contacting portion of the fabric can have holes to allow the hydrogel electrode areas to contact the patient's skin when worn. The fabric is wrapped circumferentially around the patient's head and secured using a hook and loop fastener (e.g., Velcro®) located on the outside and inside fabric surfaces, respectively. The two layers of fabric are adhered together with a narrow strip of adhesive around the complete perimeter of the device. A detachable release liner can be placed over the exposed hydrogel electrodes to prevent unintended adherence to surfaces prior to application to application on the patient.

The substrate within the fabric layers can be constructed from a stretchable substrate, such as Intexar TE-11C. The substrate may be adhered to the inner fabric layer around the hydrogel electrode area and at the perimeter of the fabric. Other areas of the substrate may be left unadhered to the fabric substrate to allow the substrate to stretch at a different rate than the fabric substrate.

The lateral electrodes may use a different fixation material from the fixation material used on the frontal electrodes. In this example, a medical-grade adhesive tape is used to secure the electrode on the patient. In this example, multiple release liners are expose different areas of the electrode without unintended adherence. This is particularly useful when a liquid electrode gel is applied over the hydrogel electrodes. The liquid electrode gel can be applied to the hydrogel electrode areas to lower the impedance of the skin interface when applied to the patient. A lower skin impedance is preferable to lower the required voltage needed to achieve a constant-current biphasic pulse of stimulation.

The device disclosed herein may comprise one or more sets of electrodes positioned in areas superior to the orbit and around and in the auricle. The ellipses and triangles shown in FIG. 1 are electrodes that may contact a subject's skin and transmit current between a pulse generator and the subject's target nerve. The electrodes may be hydrogel electrodes. The electrodes may be constructed from a conductive ink that fills the area of the electrode. The electrodes may be constructed from a composite of conductive ink with an overlying hydrogel. The hydrogel may serve to conduct current between the conductive ink and the skin, reduce impedance of this boundary, and improve the current distribution by increasing the contact area of the electrode with the skin. Multi-use high-tack hydrogels configured to enhance current transmission between a pulse generator and a respective target nerve may be suitable, for example, Axelgaard 2500 series available from Axelgaard Manufacturing Co. Ltd. of Fallbrook, CA.

The hydrogel electrodes may be positioned on a flexible substrate. The flexible substrate may comprise a thin polyethylene plastic. The flexible substrate may comprise Mylar A, for example. The flexible substrate may be printed with a conductive ink. The flexible substrate may provide structural support and spacing of the electrodes. The flexible material may be shaped to minimize the areas acting as a barrier between the adhesive and the skin. The flexible substrate may have conductive traces thereon to supply current from a pulse generator to the electrodes. The conductive ink used in the flexible substrate may be chosen to minimize impedance between the electrode and the pulse generator. A dielectric insulator may cover the conductive traces to prevent inadvertent contact with a subject's skin.

The layer surrounding and below the flexible substrate may be the adhesive layer. The adhesive layer may adhere the stimulation device to a subject's skin. The adhesive layer may be selected to be applied and removed to the skin without damaging the electrodes or the skin. The adhesive layer may be selected to allow air and moisture to penetrate its barrier to improve wearing comfort. The adhesive layer may also be selected to require a minimal amount of external force to remove so the device does not come removed from the skin during normal head movements. The adhesive layer may comprise 3M medical grade silicone adhesive tape, for example, 3M medical grade silicone adhesive 2480. The adhesive layer may be used to maintain contact between the electrodes and the skin by forming a temporary bond with a suitable area around the electrodes. The adhesive layer may adhere one or more of the forehead section, temple section, or lateral section of the device to a subject's skin. The shape of the adhesive layer may be designed to provide significant coverage over the forehead of a subject. Coverage of the forehead section can increase surface area for the stimulation device to bond to, thus reducing the chance of the device being accidentally removed or falling off. The adhesive layer may extend down behind the eyes and to the ear. The purpose of the adhesive layer may include positioning the entire stimulation device as quickly as possible. In some embodiments, the notch in the bottom middle of the adhesive should be positioned centered between the subject's eyebrows such that the lower edge of the adhesive begins to wrap to just below the upper orbit and above the eyelid. This positioning may place the lower central electrodes above the eyebrows.

With reference to FIG. 1, the device may comprise four pairs of electrodes. The left pair of electrodes may target the left auricular branch of the vagus nerve, the right pair of electrodes may target the right auricular branch of the vagus nerve, and the two center pairs may target the supraorbital trigeminal nerve branch. In some embodiments, the two medial pairs are two supraorbital electrodes sets and the two lateral pairs are two auricular electrodes sets.

### Supraorbital Electrodes Sets

The supraorbital electrodes sets comprise right supraorbital nerve location upper triangular electrode 101, left supraorbital nerve location upper triangular electrode 103, right supraorbital nerve location lower electrode 102, and left supraorbital nerve location lower electrode 104, with 101 and 102 comprising one set, and 103 and 104 comprising a second set. The supraorbital electrodes sets may be positioned above the orbit. The supraorbital electrodes sets may be positioned near and transcutaneous to branches of the supraorbital and the supratrochlear nerve branches of the trigeminal nerve. Each set of electrodes above the orbit may comprise two electrodes. The electrodes in each set may include a stimulation electrode and a return electrode. The stimulation electrode may be characterized by delivering a cathodal pulse initially during the biphasic stimulation while the return electrode may be characterized as the anodal pulse initially during biphasic stimulation. The polarity of these electrodes may be reversed in the second phase of the biphasic stimulation. In the second phase of the pulse, the return electrode can comprise the cathode. The return electrode can depolarize nerves in the surrounding area. Increasing the size of the surface area of the return electrode can reduce current density, thereby decreasing the chance that the second phase will interfere with the action potential generated in the first phase. Increasing the size of the surface area can comprise forming a larger and/or multi-sided electrode, for example, a triangular return electrode. The supraorbital electrodes comprises one set positioned superior to the left orbit and a second set of electrodes positioned superior to the right orbit. In other embodiments, there is only one set of supraorbital electrodes, positioned superior to either the left or the right orbit.

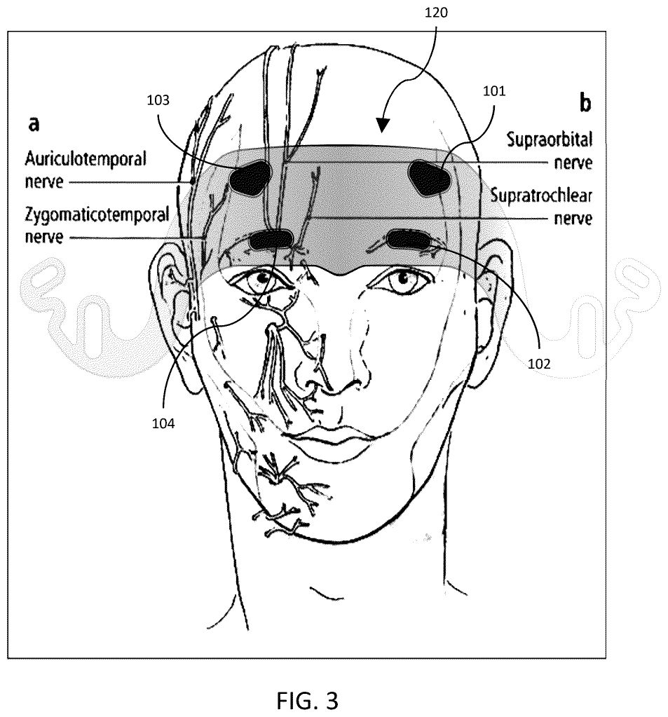

For each of the two center pairs targeting the supraorbital nerve branch, current may be transmitted from the upper triangular electrodes 101 and/or 103 to the lower electrodes 102 and/or 104. The upper triangular electrodes 101 and/or 103 may act as anodes on the initial stimulation pulse, and the lower elliptical electrodes 102 and/or 104 may act as cathodes on the initial stimulation pulse. The triangular shape of the upper central electrodes 101 and/or 103 may be designed to specifically shape the generated electric field in the orientation of the targeted nerve branch. The targeted nerve branch may be the supraorbital trigeminal nerve branch. For example, the gradient of the electrode field generated is changing in the same direction as a trajectory of the supraorbital branch of the trigeminal nerve on the forehead. This may be a benefit of the disclosed electrode assembly and/or electrode configuration in which one electrode is placed above the supraorbital foramen above the eyebrow and a second is placed higher on the forehead following the course of the nerve branch (as illustrated in FIG. 3). The larger size of the triangular electrode 101 and/or 103 relative to the other electrodes can reduce the current density at the supraorbital trigeminal nerve branch, reducing the nerve fibers that will be recruited on the secondary phase of the pulse.

Current may follow the path of least resistance. If there is constant resistance within each supraorbital electrode set, the path of least resistance can be the closest points between the two electrodes in each supraorbital electrode set. An electric field can disperse from these points. In some cases, a triangular shaped electrode with a clear protrusion can shape and concentrate the electric field. The electric field can be concentrated in the location where the supraorbital nerve branches out from the supraorbital foramen or notch. In some embodiments, the superior electrodes can be differently shaped so they are not triangular. Non-triangular superior electrodes may not be as effective. Shapes without clear protrusions may spread the electric field wider and/or weaker, so greater stimulation intensity may be needed to activate the underlying nerve bundle. Shapes without clear protrusions can comprise, for example, semicircles or ellipses, such that the inferior and superior electrodes are equidistant without a concentration point.

The lower elliptical central electrodes 102 and/or 104 may be shaped to ensure coverage of a nerve bundle above the supraorbital foramen or notch. The lower elliptical central electrodes 102 and/or 104 may be shaped to cover the supraorbital foramen. In some subjects, the foramen is not fully developed and is called the supraorbital notch. Lower elliptical central electrodes 102 and/or 104 may be located close to the overlying skin of the foramen or notch. The supraorbital foramen or notch may be a nerve bundle accounting for variability in stimulation device among subjects.

In some embodiments, the notch may refer to the width of the inferior electrodes and the spacing from the facial midline. The width may consider the location of the supraorbital foramen/notch from facial midline in different populations and account for the variance among individuals. In some embodiments, the notch may be between about 22.2 to about 33.7 mm. In some embodiments, the electrode, the flexible substrate, and the adhesive layer may be curved and configured to mimic the curvature of the orbital rim.

The lower elliptical central electrodes 102 and/or 104 may be the stimulation electrodes. The stimulation electrodes 102 and/or 104 may comprise a slight concavity that follows the approximate shape of the supraorbital ridge such that the lateral portions of the stimulation electrodes 102 and/or 104 are slightly inferior to the medial portions. The corners of the stimulation electrodes 102 and/or 104 may be rounded to prevent excessive concentration of current in any one area of the electrode.

The stimulation electrodes 102 and/or 104 may have a major axis and a minor axis. In some embodiments, the major axis is approximately between 0.25 inches to 1.5 inches in length. In some embodiments, the major axis is approximately between 0.25 inches to 0.5 inches, 0.25 inches to 0.75 inches, 0.25 inches to 1 inch, 0.25 inches to 1.25 inches, 0.25 inches to 1.5 inches, 0.5 inches to 0.75 inches, 0.5 inches to 1 inch, 0.5 inches to 1.25 inches, 0.5 inches to 1.5 inches, 0.75 inches to 1 inch, 0.75 inches to 1.25 inches, 0.75 inches to 1.5 inches, 1 inch to 1.25 inches, 1 inch to 1.5 inches, or between about 1.25 inches to 1.5 inches in length. In some embodiments, the major axis has a length of less than 0.25 inches. In some embodiments, the major axis has a length of more than 1.5 inches.

In some embodiments, the optimal length of the major axis is between about 0.5 inches to 1.25 inches. In some embodiments, the optimal length of the major axis is between about 0.5 inches to 0.75 inches, 0.5 inches to 1 inch, 0.5 inches to 1.25 inches, 0.75 inches to 1 inch, 0.75 inches to 1.25 inches, or between about 1 inch to 1.25 inches. In some embodiments, the major axis has an optimal length of less than 0.5 inches. In some embodiments, the major axis has an optimal length of more than 1.25 inches. In some embodiments, the length of the major axis is 1 inch.

The center of the major axis of the stimulation electrodes 102 and/or 104 may be slightly lateral to the supraorbital notch or foramen to align with the direction of the main nerve bundle after it exits the supraorbital notch or foramen. In some embodiments, the length of the major axis can be based on cadaveric studies of the location of the supraorbital foramen or notch from the facial midline in different populations and can account for the variance among individuals. In some embodiments, the center of the major axis is between about 0.5 inches to 2 inches lateral to the facial midline range. In some embodiments, the center of the major axis is between about 0.5 inches to 0.75 inches, 0.5 inches to 1 inch, 0.5 inches to 1.25 inches, 0.5 inches to 1.5 inches, 0.5 inches to 1.75 inches, 0.5 inches to 2 inches, 0.75 inches to 1 inch, 0.75 inches to 1.25 inches, 0.75 inches to 1.5 inches, 0.75 inches to 1.75 inches, 0.75 inches to 2 inches, 1 inch to 1.25 inches, 1 inch to 1.5 inches, 1 inch to 1.75 inches, 1 inch to 2 inches, 1.25 inches to 1.5 inches, 1.25 inches to 1.75 inches, 1.25 inches to 2 inches, 1.5 inches to 1.75 inches, 1.5 inches to 2 inches, or between about 1.75 inches to 2 inches lateral to the facial midline range. In some embodiments, the center of the major axis is less than 0.5 inches lateral to the facial midline range. In some embodiments, the center of the major axis is greater than 2 inches lateral to the facial midline range. In some embodiments, the optimal center of the major axis is between about 1 inch to 1.5 inches lateral to the facial midline range. In some embodiments, the optimal center of the major axis is between about 1 inch to 1.25 inches, 1 inch to 1.5 inches, or between 1.25 inch to 1.5 inches lateral to the facial midline range. In some embodiments, the optimal center of the major axis is less than 1 inch lateral to the facial midline range. In some embodiments, the optimal center of the major axis is greater than 1.5 inches lateral to the facial midline range. In some embodiments, the center of the major axis is 1.5 inches lateral to the facial midline range.

The stimulation electrodes 102 and/or 104 may be superior to the supraorbital notch or foramen and superior to the orbital ridge. In some embodiments, the length of the minor axis of the stimulation electrodes 102 and/or 104 is between about 0.125 inches to 1 inch. In some embodiments, the length of the minor axis is between about 0.125 inches to 0.25 inches, 0.125 inches to 0.5 inches, 0.125 inches to 0.75 inches, 0.125 inches to 1 inch, 0.25 inches to 0.5 inches, 0.25 inches to 0.75 inches, 0.25 inches to 1 inch, 0.5 inches to 0.75 inches, 0.5 inches to 1 inch, or between about 0.75 inches to 1 inch. In some embodiments, the length of the minor axis is less than about 0.125 inches. In some embodiments, the length of the minor axis is greater than about 1 inch. In some embodiments, the optimal length of the minor axis is between about 0.25 inches to 0.5 inches. In some embodiments, the optimal length of the minor axis is between about 0.25 inches to 0.375 inches, 0.25 inches to 0.5 inches, or between 0.375 inches to 0.5 inches. In some embodiments, the optimal length of the minor axis is less than about 0.25 inches. In some embodiments, the optimal length of the minor axis is greater than about 0.5 inches. In some embodiments, the length of the minor axis is approximately 0.375 inches.

The upper triangular central electrodes 101 and/or 103 may be the return electrodes of the supraorbital electrodes sets. The return electrodes 101 and/or 103 may be shaped in an approximate rounded triangular shape with one protrusion of the electrodes being positioned along an imaginary line drawn between the center of the stimulation electrodes 102 and/or 104 and the center of the return electrodes 101 and/or 103. The corners of the return electrodes 101 and/or 103 may be rounded to prevent excessive concentration of current in any one area of the electrode. The return electrodes 101 and/or 103 may be positioned superior to the stimulating electrodes. The return electrodes 101 and/or 103 may also be positioned slightly lateral to the stimulating electrodes 102 and/or 104. The positioning of the return electrodes 101 and/or 103 may approximate the course of the supraorbital branch of the trigeminal nerve when an electrical field is generated between the stimulating electrodes 102 and/or 104 and the return electrodes 101 and/or 103.

In some embodiments, the triangular shape of the superior frontal electrode is configured to reduce a current density reducing nerve fibers that will be recruited on a secondary phase of a pulse of a pulse generator. For example, in the second phase of the pulse, the superior triangular electrode can act as the cathode and depolarize nerves in the surrounding volume. By increasing the size and surface area of this electrode in a triangular shape, the current density is reduced which renders the second phase less likely to interfere (e.g., destructively interfere with the subsequent phase due to hyperpolarization disbursed away from the electrode or depolarization under the electrode) with the action potential that was generated by the initial phase. As the current between the electrodes will follow the path of least resistance, e.g., the closest distance between the electrodes assuming constant resistance between them, the electrical field will disburse from this location. The triangular shape of the electrode may modulate the electrical field generated by the electrode pair as to concentrate the electrical field in the location where the supraorbital nerve branches out from the supraorbital foramen or notch of the electrode. The triangular shape of the electrode may focus the electrical field in a narrower area as opposed to a differing electrode shape, for example a semicircle, that would instead spread the electrical field over a wider area, resulting in a reduced stimulation effect at a same stimulation intensity. A triangular shape may be beneficial in that it may not overlap with the hairline of the patient and ensure that the current will penetrate the skin as to stimulate the nerves as opposed to traveling on the surface of the skin if the electrodes were too close together.

In some embodiments, the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject. This may be a benefit of the disclosed electrode assembly and/or electrode configuration in which one electrode is placed above the supraorbital foramen above the eyebrow and a second is placed higher on the forehead following the course of the nerve branch (as illustrated in FIG. 3). In some embodiments, the triangular shape of the superior frontal electrode reduces a current density applied to nerve fibers, reducing the nerve fibers that are recruited on a secondary phase of a pulse of a pulse generator. In some embodiments, the superior frontal electrode has a triangular shape and reduces the area of an electric field produced by the electrode. In some embodiments, the triangular shape reduces the area of an electric field produced by the electrode as compared to a semicircular shaped electrode. In some embodiments, the triangular shape increases a stimulation effect configured to be applied to the subject at a same stimulation intensity. In some embodiments, the stimulation effect configured to be applied to the subject at the same stimulation intensity is increased relative to semicircular shaped electrode. In some embodiments, the triangular shape reduces off-target nerve activations when an electrical field is generated between the superior frontal electrode and the inferior frontal electrode.

The distance the return electrodes 101 and/or 103 can be positioned from the stimulating electrodes 102 and/or 104 may be constrained in many individuals by the hairline. In some embodiments, the distance between the stimulation electrodes 102 and/or 104 and return electrodes 101 and/or 103 is between about 0.25 inches to 2 inches. In some embodiments, the distance is between about 0.25 inches to 0.5 inches, 0.25 inches to 0.75 inches, 0.25 inches to 1 inch, 0.25 inches to 1.25 inches, 0.25 inches to 1.5 inches, 0.25 inches to 1.75 inches, 0.25 inches to 2 inches, 0.5 inches to 0.75 inches, 0.5 inches to 1 inch, 0.5 inches to 1.25 inches, 0.5 inches to 1.5 inches, 0.5 inches to 1.75 inches, 0.5 inches to 2 inches, 0.75 inches to 1 inch, 0.75 inches to 1.25 inches, 0.75 inches to 1.5 inches, 0.75 inches to 1.75 inches, 0.75 inches to 2 inches, 1 inch to 1.25 inches, 1 inch to 1.5 inches, 1 inch to 1.75 inches, 1 inch to 2 inches, 1.25 inches to 1.5 inches, 1.25 inches to 1.75 inches, 1.25 inches to 2 inches, 1.5 inches to 1.75 inches, 1.5 inches to 2 inches, or between about 1.75 inches to 2 inches. In some embodiments, the distance between electrodes is less than 0.25 inches. In some embodiments, the distance between electrodes is greater than 2 inches. In some embodiments, the optimal distance between stimulation electrodes 102 and/or 104 and return electrodes 101 and/or 103 is between about 0.25 inches to 2 inches. In some embodiments, the optimal distance between the stimulation electrodes 102 and/or 104 and return electrodes 101 and/or 103 is between about 0.5 inches to 1 inch. In some embodiments, the optimal distance between the stimulation electrodes 102 and/or 104 and return electrodes 101 and/or 103 is between about 0.5 inches to 0.75 inches, 0.5 inches to 1 inch, or between about 0.75 inches to 1 inch. In some embodiments, the optimal distance between electrodes is less than 0.5 inches. In some embodiments, the optimal distance between electrodes is greater than 1 inch. In some embodiments, the distance between electrodes is 0.75 inches.

The lateral spacing of the centers of the return electrodes 101 and/or 103 relative to the centers of the stimulation electrodes 102 and/or 104 may be between about 2 inches closer to the facial midline to 2 inches further away from the facial midline. In some embodiments, the lateral spacing of the centers of the return electrodes 101 and/or 103 relative to the centers of the stimulation electrodes 102 and/or 104 is between about 2 inches closer to 1 inch closer, 2 inches closer to 0 inches closer, 2 inches closer to 1 inch farther, 2 inches closer to 2 inches farther, 1 inch closer to 0 inches closer, 1 inch closer to 1 inch farther, 1 inch closer to 2 inches farther, 0 inches closer to 1 inch farther, 0 inches closer to 2 inches farther, or between 1 inch farther to 2 inches farther away from the facial midline. In some embodiments, the lateral spacing of the centers of the return electrodes 101 and/or 103 relative to the centers of the stimulation electrodes 102 and/or 104 is greater than 2 inches closer, or greater than 2 inches farther. In some embodiments, the optimal lateral spacing of the centers of the return electrodes 101 and/or 103 relative to the centers of the stimulation electrodes 102 and/or 104 is between about 0.5 inches closer to the facial midline to 0.5 inches farther away from the facial midline. In some embodiments, the optimal lateral spacing of the centers of the return electrodes 101 and/or 103 relative to the centers of the stimulation electrodes 102 and/or 104 is between about 0.5 inches closer to 0 inches closer, 0.5 inches closer to 0.5 inches farther, or between about 0 inches closer to 0.5 inches farther from the facial midline. In some embodiments, the optimal lateral spacing of the centers of the return electrodes 101 and/or 103 relative to the centers of the stimulation electrodes 102 and/or 104 is greater than 0.5 inches closer, or greater than 0.5 inches farther. In some embodiments, the lateral spacing of the centers of the return electrodes 101 and/or 103 relative to the centers of the stimulation electrodes 102 and/or 104 is approximately 0.25 inches to 0.5 inches farther away from the facial midline.

### Auricular Electrodes Sets

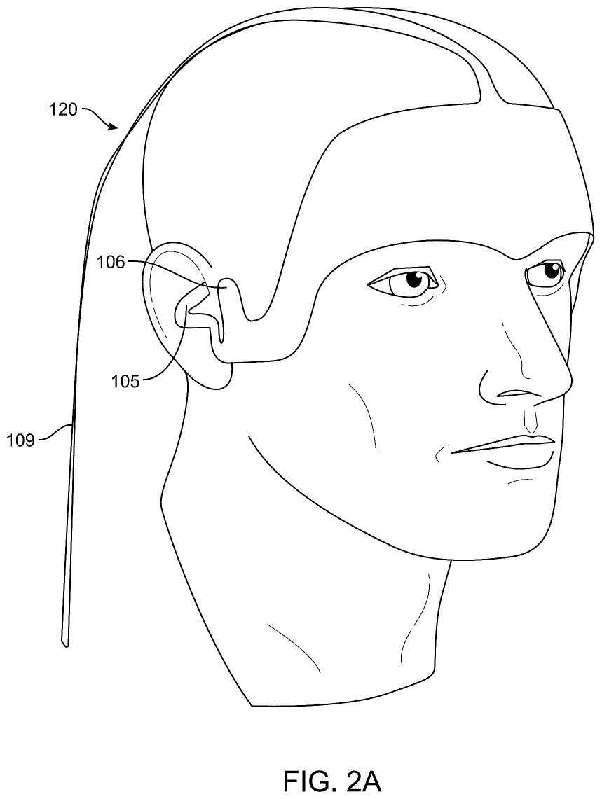

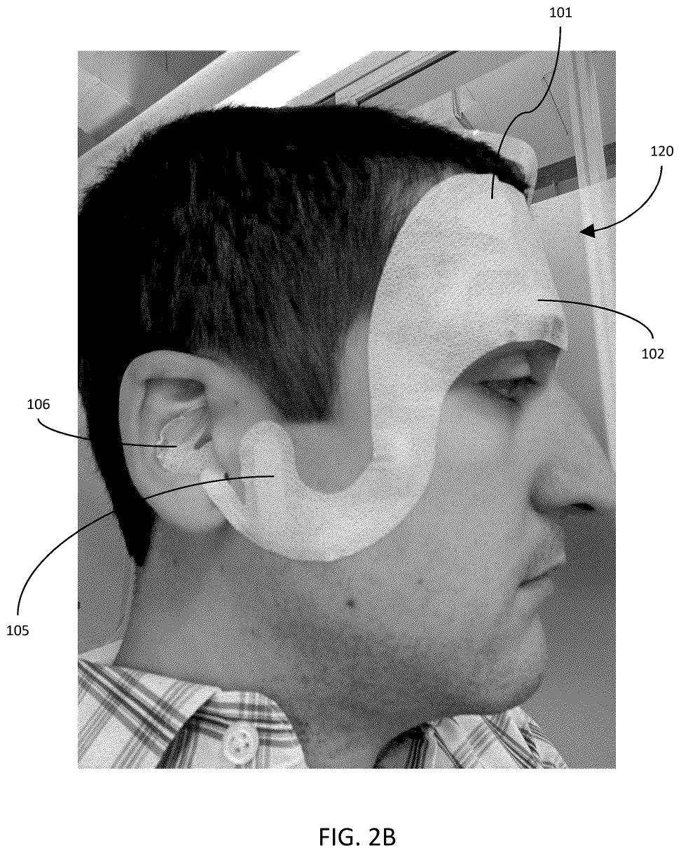

FIGS. 1-2B show an exemplary stimulation device 120 for neural stimulation of a plurality of target nerves in a subject. In some embodiments, the auricular electrodes sets comprise right vagus nerve location larger elliptical electrode 105, left vagus nerve location larger elliptical electrode 107, right vagus nerve location smaller elliptical electrode 106, and left vagus nerve location smaller elliptical electrode 108, with 105 and 106 comprising one set, and 107 and 108 comprising a second set. The electrodes positioned in and around the auricle may be positioned near and transcutaneous to branches of the auricular branch of the vagus nerve and the auriculotemporal branch of the trigeminal nerve. Each set of electrodes positioned in and around the auricle may comprise two electrodes: a stimulation electrode and a return electrode. The stimulation electrodes may be characterized by delivering a cathodal pulse initially during the biphasic stimulation while the return electrodes may be characterized as the anodal pulse initially during biphasic stimulation. The polarity of these electrodes may be reversed in the second phase of the biphasic stimulation. In some embodiments, the auricular electrodes comprises one set positioned in and around the left auricle and a second set of electrodes positioned in and around the right auricle. In other embodiments, there is only one set of auricular electrodes, positioned in and around either the left or the right auricle.

The left and right pairs of electrodes targeting the vagus nerve may have current transmitted from the larger electrodes 105 and/or 107 to the smaller electrodes 106 and/or 108. The larger electrodes 105 and/or 107 may be positioned in front of the tragus and the smaller electrodes 106 and/or 108 are positioned over the cymba concha in the ear. The larger electrodes 105 and/or 107 may act as anodes on the initial stimulation pulse, and the smaller electrodes 106 and/or 108 may act as cathodes on the initial stimulation pulse.

The smaller electrodes 106 and/or 108 may be the stimulation electrodes. If the stimulation device 120 were laid flat as shown in FIG. 1, the positioning of the auricular stimulation electrodes 106 and/or 108 may be slightly below the supraorbital stimulation electrodes 102 and/or 104. When positioned on the subject, the auricular stimulation electrodes 106 and/or 108 may be designed to overlie the cymba concha of the outer ear, inferior to the crura of the antihelix and superior to the crus of the helix. The corners of the stimulation electrodes 106 and/or 108 may be rounded to prevent excessive concentration of current in any one area of the electrodes. The stimulation electrodes 106 and/or 108 may be positioned within their respective cymba conchas as closely as possible onto the overlying skin of the respective auricular branches of the left and right vagus nerves. There may be anatomic variations among subjects that can be accounted for through ranges of electrode dimensions. In some embodiments, a portion of the substrate connecting the larger electrodes 105 and/or 107 to the smaller electrodes 106 and/or 108 has an arc shape 110.

The stimulation electrodes 106 and/or 108 may have a major axis and a minor axis. In some embodiments, the length of the major axis is between about 0.25 inches to 1.5 inches. In some embodiments, the length of the major axis is approximately between 0.25 inches to 0.5 inches, 0.25 inches to 0.75 inches, 0.25 inches to 1 inch, 0.25 inches to 1.25 inches, 0.25 inches to 1.5 inches, 0.5 inches to 0.75 inches, 0.5 inches to 1 inch, 0.5 inches to 1.25 inches, 0.5 inches to 1.5 inches, 0.75 inches to 1 inch, 0.75 inches to 1.25 inches, 0.75 inches to 1.5 inches, 1 inch to 1.25 inches, 1 inch to 1.5 inches, or between about 1.25 inches to 1.5 inches. In some embodiments, the major axis has a length of less than 0.25 inches. In some embodiments, the major axis has a length of more than 1.5 inches. In some embodiments, the optimal length of the major axis is between about 0.375 inches to 1 inch. In some embodiments, the optimal length of the major axis is between about 0.375 inches to 0.5 inches, 0.375 inches to 0.625 inches, 0.375 inches to 0.75 inches, 0.375 inches to 0.875 inches, 0.375 inches to 1 inch, 0.5 inches to 0.625 inches, 0.5 inches to 0.75 inches, 0.5 inches to 0.875 inches, 0.5 inches to 1 inch, 0.625 inches to 0.75 inches, 0.625 inches to 0.875 inches, 0.625 inches to 1 inch, 0.75 inches to 0.875 inches, 0.75 inches to 1 inch, or between about 0.875 inches to 1 inch. In some embodiments, the major axis has an optimal length of less than 0.375 inches. In some embodiments, the major axis has an optimal length of more than 1 inch. In some embodiments, the major axis has a length of 0.53 inches.

Relative to the facial midline if the device 120 were laid flat as shown in FIG. 1, the centers of stimulation electrodes 106 and/or 108 may be lateral to the supraorbital electrodes sets 110. In some embodiments, the distance of the centers of the stimulation electrodes 106 and/or 108 from the facial midline range is between about 4.5 inches to 8.5 inches. In some embodiments, the distance of the centers of the stimulation electrodes 106 and/or 108 from the facial midline range is between about 4.5 inches to 5.5 inches, 4.5 inches to 6.5 inches, 4.5 inches to 7.5 inches, 4.5 inches to 8.5 inches, 5.5 inches to 6.5 inches, 5.5 inches to 7.5 inches, 5.5 inches to 8.5 inches, 6.5 inches to 7.5 inches, 6.5 inches to 8.5 inches, or between about 7.5 inches to 8.5 inches. In some embodiments, the distance of the centers of the stimulation electrodes 106 and/or 108 from the facial midline range is less than about 4.5 inches. In some embodiments, the distance of the centers of the stimulation electrodes 106 and/or 108 from the facial midline range is greater than about 8.5 inches. In some embodiments, the optimal distance of the centers of the stimulation electrodes 106 and/or 108 from the facial midline range is between about 5 inches to 8 inches. In some embodiments, the optimal distance of the centers of the stimulation electrodes 106 and/or 108 from the facial midline range is between about 5 inches to 6 inches, 5 inches to 7 inches, 5 inches to 8 inches, 6 inches to 7 inches, 6 inches to 8 inches, or between about 7 inches to 8 inches. In some embodiments, the optimal distance of the centers of the stimulation electrodes 106 and/or 108 from the facial midline range is less than about 5 inches. In some embodiments, the optimal distance of the centers of the stimulation electrodes 106 and/or 108 from the facial midline range is greater than about 8 inches. In some embodiments, the distance of the centers of the stimulation electrodes 106 and/or 108 from the facial midline range is approximately 6.5 inches.

The length of the minor axis of the auricular stimulation electrodes 106 and/or 108 may be sized to have sufficient area to produce a consistent and tolerable current density while maintaining a size small enough to fit and target the cymba concha. In some embodiments, the length of the minor axis is between about 0.0625 inches to 1 inch. In some embodiments, the length of the minor axis is between about 0.0625 inches to 0.25 inches, 0.0625 inches to 0.5 inches, 0.0625 inches to 0.75inches, 0.0625 inches to 1 inch, 0.25 inches to 0.5 inches, 0.25 inches to 0.75 inches, 0.25 inches to 1 inch, 0.5 inches to 0.75 inches, 0.5 inches to 1 inch, or between about 0.75 inches to 1 inch. In some embodiments, the length of the minor axis is less than about 0.0625 inches. In some embodiments, the length of the minor axis is greater than about 1 inch. In some embodiments, the optimal length of the minor axis is between about 0.125 inches to 0.5 inches. In some embodiments, the optimal length of the minor axis is between about 0.125 inches to 0.25 inches, 0.125 inches to 0.375 inches, 0.125 inches to 0.5 inches, 0.25 inches to 0.375 inches, 0.25 inches to 0.5 inches, or between about 0.375 inches to 0.5 inches. In some embodiments, the optimal length of the minor axis is less than about 0.125 inches. In some embodiments, the optimal length of the minor axis is greater than about 0.5 inches. In some embodiments, the length of the minor axis is 0.25 inches.

The larger electrodes 105 and/or 107 may be the return electrodes. The corners of the return electrodes 105 and/or 107 may be rounded to prevent excessive concentration of current in any one area of the electrodes. The return electrodes of the auricle electrodes sets 105 and/or 107 may be positioned slightly inferior to the stimulating electrodes 106 and/or 108. The return electrodes 105 and/or 107 may also be positioned slightly medial to the stimulating electrodes 106 and/or 108 if the device 120 were laid flat. The return electrodes 105 and/or 107 may overlie the skin of the each auriculotemporal branch of the trigeminal nerves in an area in front of each tragus. The positioning of the return electrodes 105 and/or 107 can minimize off-target nerve activations when an electrical field is generated between the stimulating electrodes 106 and/or 108 and the return electrodes 105 and/or 107.

In some embodiments, the set of lateral electrodes generates an electrical field in an orientation of an auricular branch of the vagus nerve. In some embodiments, the orientation of the electrical field is parallel to one or more nerve fibers in the auricular branch of the vagus nerve. In some embodiments, the substrate comprises a frontal section comprising the set of frontal electrodes a lateral section comprising to the set of lateral electrodes, wherein the lateral section is removable from the frontal section. In some embodiments, the substrate is unitary and continuous between the frontal section and the lateral section. In some embodiments, the frontal section, is detachable from the lateral section. In some embodiments, the electrode assembly comprises the set of frontal electrodes and the set of lateral electrodes. In some embodiments, the set of lateral electrodes are removable from the device. In some embodiments, each electrode in the first set of electrodes is in independent communication with at least one of the plurality of electrical connections the electrical connection. In some embodiments, a position of the concha electrode and the tragus electrode minimize off-target nerve activations when an electrical field is generated between the concha electrode and the tragus electrode and applied to the subject.

The distance the return electrode 105 and/or 107 can be positioned from the stimulating electrode may be constrained in many subjects by the sideburns and the auricles. The return electrodes 105 and/or 107 may be sized to be approximately double the area of the stimulation electrodes. The return electrodes 105 and/or 107 may have a major axis and a minor axis. In some embodiments, the major axis of return electrodes 105 and/or 107 is approximately between 0.25 inches to 1.5 inches in length. In some embodiments, the major axis is approximately between 0.25 inches to 0.5 inches, 0.25 inches to 0.75 inches, 0.25 inches to 1 inch, 0.25 inches to 1.25 inches, 0.25 inches to 1.5 inches, 0.5 inches to 0.75 inches, 0.5 inches to 1 inch, 0.5 inches to 1.25 inches, 0.5 inches to 1.5 inches, 0.75 inches to 1 inch, 0.75 inches to 1.25 inches, 0.75 inches to 1.5 inches, 1 inch to 1.25 inches, 1 inch to 1.5 inches, or between about 1.25 inches to 1.5 inches in length. In some embodiments, the major axis has a length of less than 0.25 inches. In some embodiments, the major axis has a length of more than 1.5 inches. In some embodiments, the optimal length of the major axis is between about 1 inch to 1.5 inches. In some embodiments, the optimal length of the major axis is between about 1 inch to 1.25 inches, 1 inch to 1.5 inches or between about 1.25 inches to 1.5 inches. In some embodiments, the major axis has an optimal length of less than 1 inch. In some embodiments, the major axis has an optimal length of more than 1. 5 inches. In some embodiments, the length of the major axis is approximately 1.125 inches.

In some embodiments, the length of the minor axis of the return electrodes 105 and/or 107 is between about 0.125 inches to 1 inch. In some embodiments, the length of the minor axis is between about 0.125 inches to 0.25 inches, 0.125 inches to 0.5 inches, 0.125 inches to 0.75 inches, 0.125 inches to 1 inch, 0.25 inches to 0.5 inches, 0.25 inches to 0.75 inches, 0.25 inches to 1 inch, 0.5 inches to 0.75 inches, 0.5 inches to 1 inch, or between about 0.75 inches to 1 inch. In some embodiments, the length of the minor axis is less than about 0.125 inches. In some embodiments, the length of the minor axis is greater than about 1 inch. In some embodiments, the optimal length of the minor axis is between about 0.25 inches to 0.5 inches. In some embodiments, the optimal length of the minor axis is between about 0.25 inches to 0.375 inches, 0.25 inches to 0.5 inches, or between about 0.375 inches to 0.5 inches. In some embodiments, the optimal length of the minor axis is less than about 0.25 inches. In some embodiments, the optimal length of the minor axis is greater than about 0.5 inches. In some embodiments, the length of the minor axis is approximately 0.375 inches.

FIG. 2 provides a front-right view of a stimulation device 120 as worn on a subject's head. In some embodiments, with reference to FIG. 2A, the stimulation device 120 comprises a forehead section extending to the ears and a tail section comprising a reinforced interface for an external pulse generator connection 109. In some cases, with reference to FIG. 2B, the placement of the stimulation device 120 surrounds the transcranial temporal window. The stimulation device 120 on the forehead may lie below the hairline and over the eyebrows. A tail of the stimulation device 120 may comprise the conductive traces of the electrodes. The tail may extend over the top of a subject's head. The tail may comprise the flexible substrate but may not have the adhesive layer outside of the perimeter of the flexible substrate. The side of the adhesive layer comprising the electrodes may be facing the subject's skin, and the empty side of the adhesive layer may be facing away from the subject's skin.

The shape of the device may provide sufficient area for the adhesive layer to bond to the skin in areas not overlying the electrodes or the flexible circuit. The shape of the device 120 may minimize application of the adhesive layer to areas of hair of the user, such as the eyebrows, scalp, and/or sideburns. In some embodiments, the shape of the device 120 may not cover the temporal bone and leaves open ultrasonic windows to allow for transcranial Doppler ultrasound assessment of the cerebral arteries without removal of the device 120. For example, the window may be configured such that a 2 MHz TCD ultrasound transducer can be placed over the temporal area just above the zygomatic arch and in front of the tragus of the ear, permitting the user to orient the transducer slightly upward, anteriorly. In some embodiments, the shape of the device 120 may not overlie the eyes of the user or prevent assessment of facial droop of the user. In some embodiments, the device materials may not be rigid, so the device 120 can be shaped to accommodate the facial anatomy of the user and allow for slight adjustments to ensure the electrodes overlie the intended nerve targets.

In some embodiments, the stimulation device 120 described herein surrounds but leaves open the transcranial temporal window. The transcranial temporal window can be used for transcranial Doppler ultrasound assessment of cerebral arteries of a subject. Transcranial Doppler ultrasound assessment can be used without removal of the stimulation device 120. Transcranial Doppler ultrasound assessment can be used before, during, and/or after stimulation from the stimulation device 120.

A transcranial temporal window can be a collection of four windows or tiny openings where sound waves can enter and exit the skull. The transcranial temporal window can be located in the transtemporal region of the skull. The four windows can comprise a middle, posterior, anterior, and frontal windows. In some embodiments, with reference to FIG. 2B, the rostral light circle comprises an anterior transcranial temporal window, the middle light circle comprises a middle transcranial temporal window, and the caudal light circle comprises a posterior transcranial temporal window.

Transcranial temporal windows can be easy or difficult to find depending on one or more of age, race, and gender. In some embodiments, a method of finding the transcranial temporal windows can be to begin on one side of the head and continue to the other side with a transducer until the most robust middle cerebral artery (MCA) signal is found. Finding the strength of the MCA signal can depend on sound and visual density. In other embodiments, a method of finding the transcranial temporal windows can comprise placing a transducer over the temporal area just above the zygomatic arch and in front of the tragus of the ear and orient the transducer slightly upward in the anterior direction. The transducer can be a 2 MHz transcranial ultrasound transducer.

In some embodiments, a method of opening the transcranial temporal window(s) can be to have a subject open and clench their mouth repeatedly while another person places their hand over the subject's temple. The area with the most flesh or muscle concentration can be the location of the thinnest bone density. The location of thinnest bone density can be a landmark for locating the transcranial temporal window(s). Once the transcranial temporal window is found, the location can be marked, for example, marked with a dot or an arrow. The mark can be applied with surgical pen so the mark does not rub off easily.

FIG. 3 provides a front view of a subject wearing an exemplary stimulation device 120, with underlying nerves shown. The frontal section of the stimulation device 120 can be worn over the forehead of a subject. The frontal section of the stimulation device 120 can lie, in part or in whole, over one or more of an auriculotemporal nerve, a zygomaticotemporal nerve, a supraoribital nerve, and a supratrochlear nerve. The frontal section of the stimulation device 120 can lie, in part or in whole, over one set of nerves on either side of the face. The frontal section of the stimulation device 120 can lie, in part or in whole, over two sets of nerves on both sides of the face. The frontal section of the stimulation device 120 can lie, in part or in whole, over additional nerves in the head and/or face.

In some embodiments, supraorbital nerve location upper triangular electrodes 103 and/or 101 can lie, in part or in whole, over one or more nerves. The one or more nerves can include one or more supraorbital nerves. In some embodiments, supraorbital nerve location lower electrode 104 and/or 102 can lie, in part or in whole, over one or more nerves. The one or more nerves can include one or more supraorbital nerves. In some embodiments, stimulation of the one or more supraorbital nerves may also stimulate one or more supratrochlear nerves.

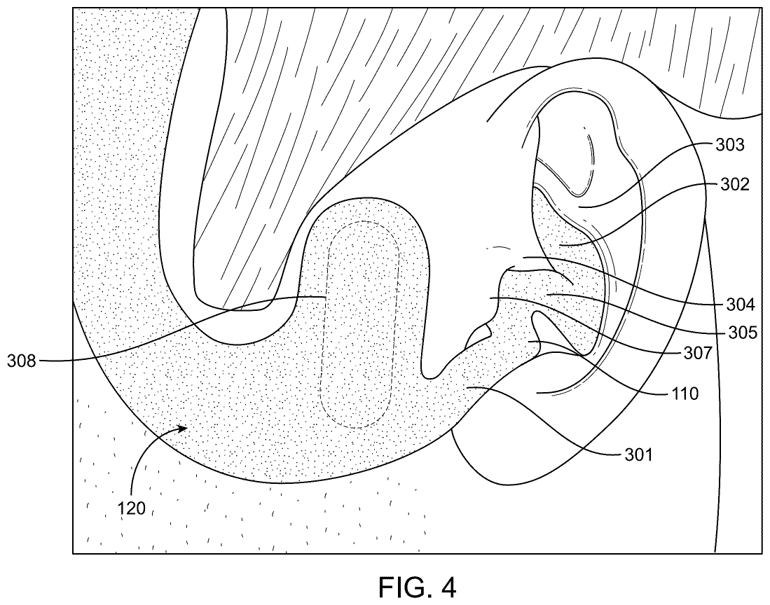

FIG. 4 provides a side view of a subject's left ear wearing an exemplary stimulation device 120. The part of the stimulation device 120 comprising the smallest electrode may be located in the cymba concha 302 and may fit snugly in the space below the antihelix 303 and above the helicis crus 304. The adhesive tape around the hydrogel electrode may promote further adhesion. The flexible substrate and adhesive layer may have an arc shape 110 to follow the contour of the helicis crus and increase surface area for adhesion within the cavum concha 305. The stimulation device 120 may exit the outer ear at the intertragic notch 301. The larger electrode on the left side may be positioned in front of the tragus 307 and behind the hair of the sideburn 308. The side of the adhesive layer comprising the electrodes may be facing the subject's skin, and the empty side of the adhesive layer may be facing away from the subject's skin.

In some embodiments, knowing the anatomy of the external ear is important in properly applying the stimulation device 120. The external ear can comprise a helix, triangular fossa, antihelix crura, scaphoid fossa, antihelix, helix crus, tragus, antitragus, intertragic notch, lobule, cymba, cavum, and concha.

The helix is the outermost part of the ear. The helix is the outer rim of the ear that extends from the superior insertion of the ear on the scalp to the termination of the cartilage at the earlobe. The lower portion is often non-cartilaginous. The border of the helix usually forms a rolled rim but the helix is variable in shape.

The triangular fossa is a concave area bounded by the antihelix crura and the ascending portion of the helix closest to the superior insertion of the ear on the scalp.

The antihelix crura comprises an inferior and a superior portion. The superior portion is an upper cartilaginous ridge arising out of the bifurcation of the antihelix 303 at near the superior part of the ear. The superior portion runs in a superior and slightly anterior direction and is usually less sharply folded than the lower portion. The inferior portion is a lower cartilaginous ridge arising out of the bifurcation of the antihelix 303 that ends beneath the fold of the ascending helix and separates the concha from the triangular fossa. The inferior portion runs in an anterior and slightly superior direction, can be sharply defined, and appears less variable than the superior portion.

The antihelix 303 is Y-shaped curved cartilaginous ridge arising out of the antitragus and separating the concha, triangular fossa, and scaphoid fossa. The antihelix 303 is a folding of the conchal cartilage and usually has similar prominence to a well-developed helix. The part of the antihelix 303 below the bifurcation is slightly curved and branches about two thirds of the way along its course to the superior part of the ear to form the broad fold of the superior and inferior antihelix crura.

The scaphoid fossa is the groove between the helix and the antihelix, located on the caudal part of the ear. The helix crus 304 is a ridge separating the cymba and cavum of the concha in the inner part of the external ear. The helix crus 304 can extend about one-half to two-thirds the distance across the concha and extends in the rostral-caudal direction. The tragus 307 is a rostral area of skin-covered cartilage located halfway down the junction between the head and the ear. The tragus 307 is located across from and rostral to the antitragus. The antitragus is a cartilaginous ridge lying between the inferior part of the ear and the origin of the antihelix 303. The antitragus is located across from and caudal to the tragus 307. The intertragic notch 301 is located where the tragus 307 meets the antitragus, on the inferior portion of the ear above the lobule.

The lobule is a fleshy part of the ear on the inferior part of the ear, near the connection of the inferior part of the ear to the head. The lobule is non-cartilaginous.

The concha is bounded by the antihelix 303, inferior antihelix crura, antitragus, tragus 307, and intertragic notch 301. The concha is the innermost part of the external ear. The concha is bisected by the helix crus. The concha comprises the cymba 302 and the cavum 305. The cymba 302 is the superior portion of the concha, located above the helix crus. The cavum 305 is the inferior portion of the concha, located below the helix crus 304. The inner part of the cavum 305 connects to the middle ear.

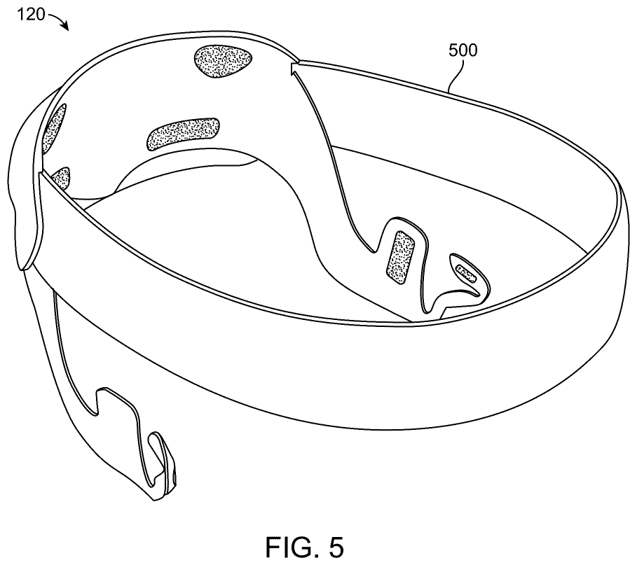

FIG. 5 provides a rear perspective view depiction of an exemplary stimulation device 120. The stimulation device 120 can be used with an external fixation device 500. The fixation device 500 may be an elastic headband or frame over the stimulation device 120. The stimulation device 120 may be used in conjunction with a rigid external frame. The fixation device 500 may allow for additional adhesion and contact with the subject's skin. The external frame may overlie the electrodes of the stimulation device 120 and may apply a force to the stimulation device 120 in the direction of the skin. This force can have the effect of minimizing the thickness of skin and underlying dermal tissue, fat, and/or muscle separating the electrode and the target nerve. In some embodiments, the fixation device 500 puts pressure over the electrodes to increase contact with the subject's skin and decrease the distance to the target nerves. The fixation device 500 may decrease impedance of the stimulation device 120 and skin interface. The fixation device 500 may decrease the chance of any potential negative side effects of stimulation. Potential negative side effects of stimulation that are reduced by the fixation device 500 include, but are not limited to, burning, tingling, pain, numbness, soreness, bruising, or any combination thereof. The side of the stimulation device 120 comprising the electrodes may be facing the subject's skin, and the empty side of the stimulation device 120 may be facing away from the subject's skin.

The frame may be constructed with a rigid front that overlies the positions of the electrodes and a flexible head strap that allows attachment of the stimulation device to the head of the subject. An additional headstrap may be positioned superiorly over the head of the subject between the ears to prevent the external frame from moving inferiorly. The external frame may be injection molded and constructed from a suitable plastic including, but not limited to, polypropylene. The external frame may have adjustable components that allow repositioning of the portions in contact and applying force to the electrodes. A force applied to the electrodes may be generated as a result of the material properties of the headframe and the geometry that causes an interference fit between the head frame and the electrodes overlying the subject's skin. A force may be generated between the adjustable components and the rest of the headframe using a spring, such as a torsion spring. The spring may have a spiral-shaped configuration. The spiral may be a shape with windings about a central axis. The windings may gradually widen or tighten along the length. The spiral may be continuous. The spring may have a conical-shaped deployed configuration including, but not limited to, tubular, conical, frustoconical, or helical shapes.

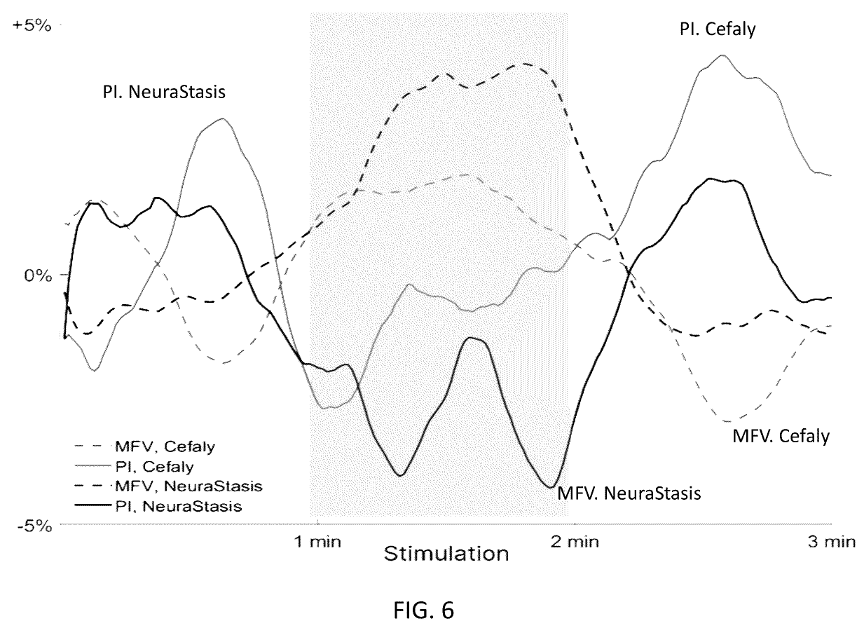

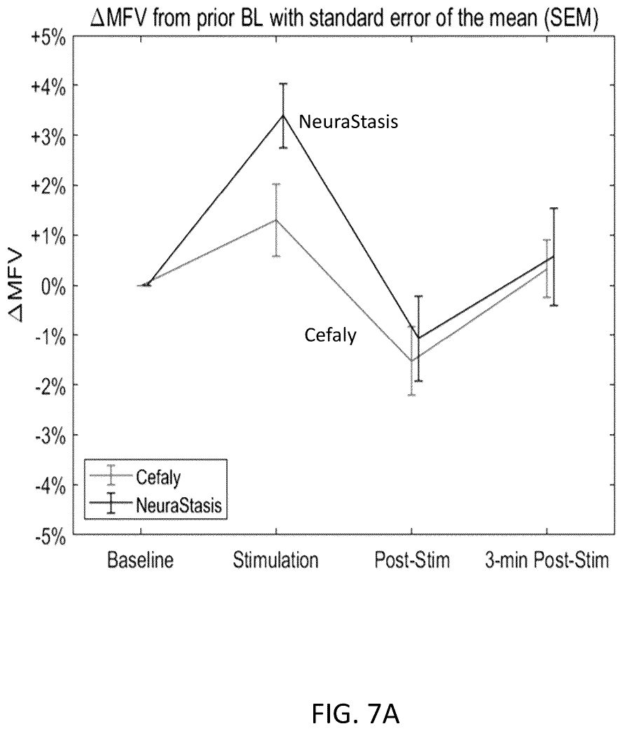

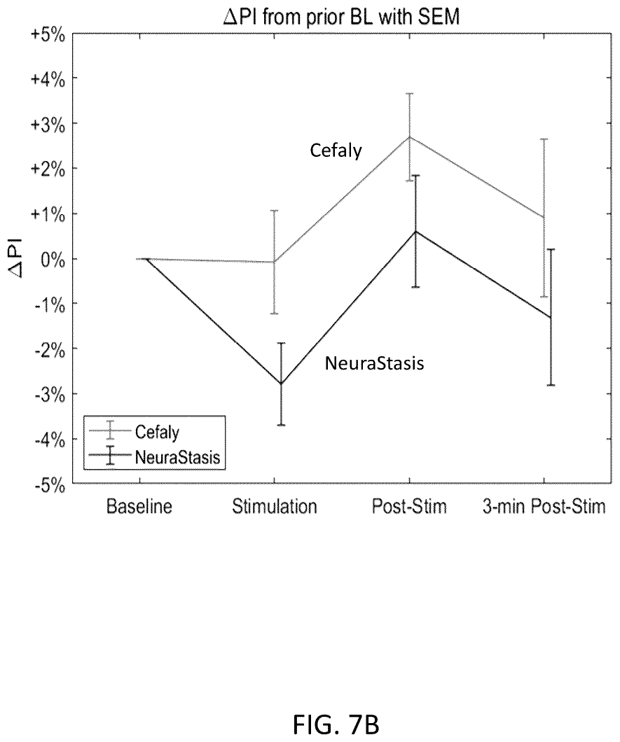

FIGS. 6, 7A, and 7B provide an exemplary depiction of mean flow velocity and pulsatility index resulting from stimulation from an embodiment of a stimulation device disclosed herein compared to similar stimulation using electrodes of an existing device available in the market, particularly the CEFALY® system available from Cefaly Technology of Seraing, Liege, Belgium. Stimulation from the device according to the disclosure herein produces a greater increase in mean flow velocity and a greater reduction in pulsatility index than that from the CEFALY® device electrodes. Pulsatility index is a measure of vascular resistivity. The combination of mean flow velocity and pulsatility index measurements may indicate increased cerebral blood flow. The changes in mean flow velocity and pulsatility index are measured using transcranial Doppler ultrasound monitoring. The stimulation parameters are the same between the device disclosed herein and the existing device. The stimulation period used to collect the data in the graphs is 1 minute. With reference to FIG. 6, the lighter line comprises the pulsatility index from stimulation by the CEFALY® device electrodes, the lighter dotted line comprises the mean flow velocity from stimulation by the CEFALY® device electrodes, the darker line comprises the pulsatility index of the device according to the disclosure herein, and the darker dotted line comprises the mean flow velocity of the device according to the disclosure herein. This graph is a time series averaged across nine trials for each stimulation device. The y-axis of the graph shows percent change, and the x-axis shows time, with stimulation occurring between minute 1 and 2 for the duration of 1 minute. The stimulation duration is indicated by the shaded region. In some embodiments, a larger positive change in mean flow velocity is indicative of increased cerebral blood flow and effectiveness of the stimulation device. A larger negative change in pulsatility may be indicative of increased cerebral blood flow and effectiveness of the stimulation device. The device described herein had a larger positive change in mean flow velocity and a larger negative change in pulsatility during the stimulation period, thus suggesting the effectiveness of the device according to the disclosure herein for treating ischemic stroke via increased cerebral blood flow may be greater than existing devices. After the cessation of stimulation, stimulation from the CEFALY® device electrodes produced a larger increase in MFV and more negative response in PI than the device described herein, which more closely resembled the pre-stimulation baseline.

With reference to FIG. 7A, the depiction of change in mean flow velocity from a prior baseline with a standard error of the mean is the lighter line for the CEFALY® device electrodes-based stimulation and the darker line for stimulation using the device according to the disclosure herein. This graph comprises average values across nine trials for each stimulation device. The y-axis shows change in mean flow velocity as a percent of the baseline, and the x-axis shows four time points comprising a baseline time point, a stimulation time point, a post-stimulation time point, and a 3-minutes-post-stimulation time point. A larger positive change in mean flow velocity may be indicative of increased cerebral blood flow and effectiveness of the stimulation device. The device according to the disclosure herein had a larger positive change in mean flow velocity during the stimulation period and after the stimulation period both immediately and three minutes after, with (p<0.05), thus suggesting the effectiveness of the device described herein for treating ischemic stroke via increased cerebral blood flow may be greater than existing devices.

With reference to FIG. 7B, the depiction of change in pulsatility index from a prior baseline with a standard error of the mean is lighter line for the CEFALY® device electrodes-based stimulation and a darker line for stimulation using the device according to the disclosure herein. This graph comprises average values across nine trials for each stimulation device. The y-axis shows change in pulsatility index as a percent of the baseline, and the x-axis shows four time points comprising a baseline time point, a stimulation time point, a post-stimulation time point, and a 3-minutes-post-stimulation time point. A larger negative change in pulsatility may be indicative of increased cerebral blood flow and effectiveness of the stimulation device. The device described herein had a larger negative change in pulsatility during the stimulation period and after the stimulation period both immediately and three minutes after, with (p<0.05), thus suggesting the effectiveness of the device described herein for treating ischemic stroke via increased cerebral blood flow may be greater than existing devices.

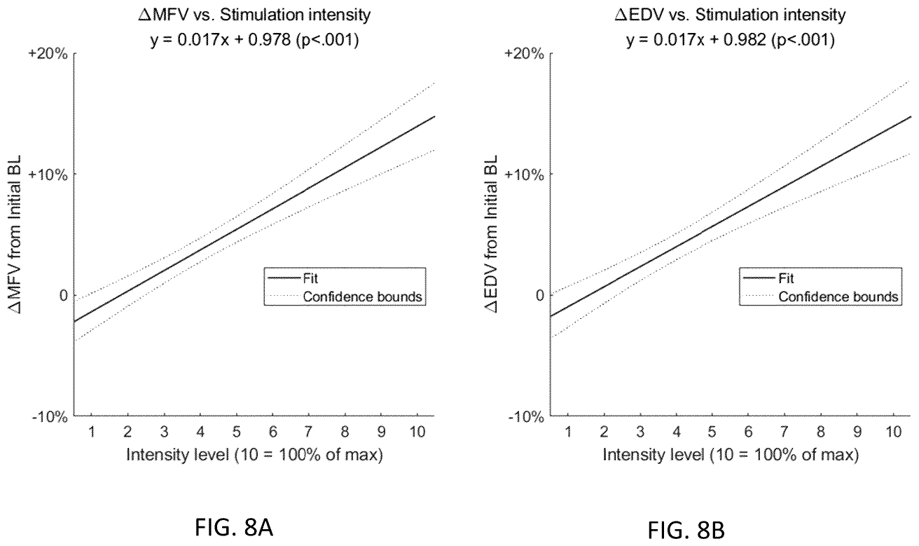

FIGS. 8A and 8B provide an exemplary depiction of mean flow velocity and end diastolic velocity measured at M1 segment of bilateral middle cerebral arteries resulting from stimulation from an embodiment of a stimulation device disclosed herein. Stimulation from the device according to the disclosure herein produces a significant increase in mean flow velocity and end diastolic velocity. End diastolic velocity has been associated with early neurological improvement and better functional outcomes in acute ischemic stroke patients. The changes in flow velocities and pulsatility index are measured using transcranial doppler ultrasound monitoring. The data in the graphs was collected during a variable-duration tolerability assessment, during which the amplitude of electrical stimulation (or intensity) delivered was varied to a submaximal level of tolerability. Mean flow velocity and end diastolic velocity were assessed at each intensity level. In both figures, each set of colored dots represents the response from a single participant, which has been normalized to the respective average of mean flow velocity and end diastolic velocity obtained during a 10-minute pre-stimulation baseline period. The y-axis of the graph shows percent change, and the x-axis shows different stimulation intensity levels. In some embodiments, a larger positive change in both mean flow velocity and end diastolic velocity are indicative of greater cerebral blood perfusion during stimulation and effectiveness of the stimulation device. The device described herein had a larger positive change in mean flow velocity and a larger negative change in pulsatility during the stimulation period, thus suggesting the effectiveness of the device according to the disclosure herein for treating neurological conditions with decreased cerebral perfusion via increased cerebral blood flow may be greater than existing devices.

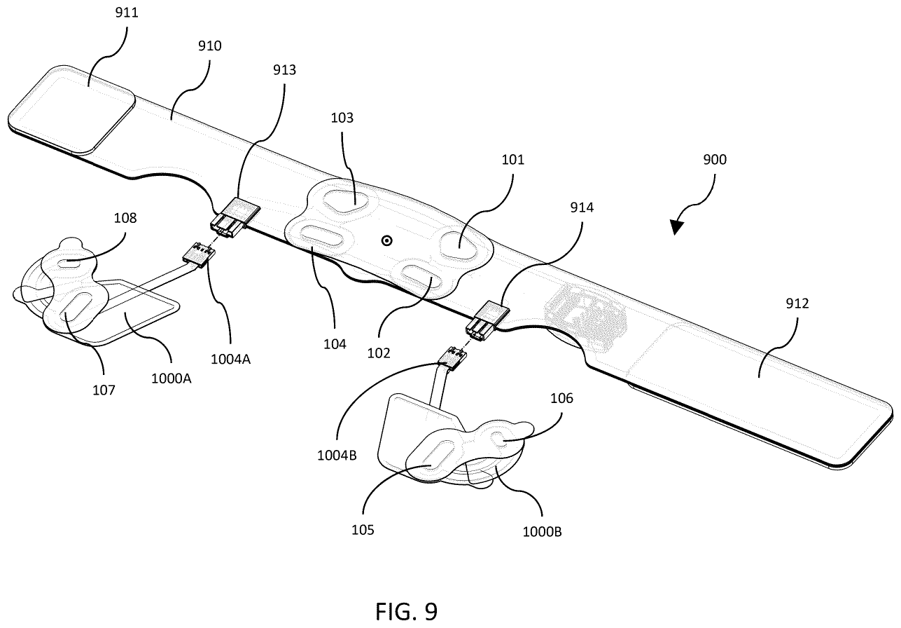

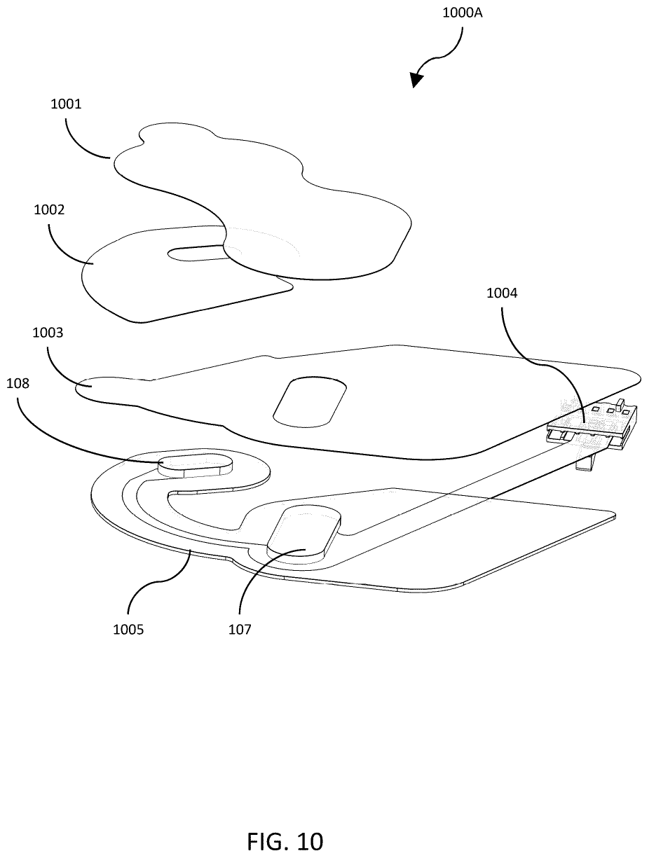

FIGS. 9 and 10 shows an exemplary device 900 comprising a frontal section 910, a left lateral section 1000A, and a right lateral section 1000B. In some embodiments, as shown, left lateral section 1000A, and a right lateral section 1000B are detachable from the frontal section. In some embodiments, as shown, the right lateral section and the left lateral section can be reattached to from the frontal section 910. In some embodiments, as shown, left lateral section 1000A, and a right lateral section 1000B are removably and electrically coupled to the frontal section 910.

Further, as shown, the frontal section 910 comprises the right supraorbital nerve location upper triangular electrode 101, the left supraorbital nerve location upper triangular electrode 103, the right supraorbital nerve location lower electrode 102, and the left supraorbital nerve location lower electrode 104. In some embodiments, the left lateral section 1000A comprises the left vagus nerve location larger elliptical electrode 107 and the left vagus nerve location smaller elliptical electrode 108, wherein the right lateral section 1000B comprises the right vagus nerve location larger elliptical electrode 105 and the right vagus nerve location smaller elliptical electrode 106.

As shown, the frontal section 910 comprises a left frontal detachable connection 913 removably couplable to a left lateral detachable connection 1004A, and a right frontal detachable connection 914 removably couplable to a right lateral detachable connection 1000B. In some embodiments, the left frontal detachable connection 913, the left lateral detachable connection 1004A, the right frontal detachable connection 914, the right lateral detachable connection 1000B, or any combination thereof comprise a connector housing (e.g., a MEMCON MHL connector).

In some embodiments, the frontal section 910 is formed of a stretchable fabric headband. In some embodiments, the frontal section 910 comprises a hook and loop fasteners 911 912 (e.g., Velcro®) to attach the headband around a patient's head. The frontal electrodes 101 102 103 104 are shown to penetrate the inner fabric layer and a release liner is shown to cover the hydrogel electrodes. The lateral electrodes 105 106 107 108 are further shown to be detachable from the frontal electrodes 101 102 103 104 at connectors 913 914 located on the perimeter of the fabric. The lateral electrodes 101 102 103 104 may use an adhesive tape fixation. The adhesive tape and hydrogel electrode areas may be covered by multiple release liners.

FIG. 10 provides an isometric exploded view of an exemplary left lateral section 1000A. In some embodiments, the left lateral section 1000A comprises the left vagus nerve location larger elliptical electrode 107, the left vagus nerve location smaller elliptical electrode 108 and the left lateral detachable connection 1004A. In some embodiments, multiple release liners 1001 1002 1003 are used to aid in application of electrode gel and application of the electrode to the patient. The top release liner 1001 is used to cover exposure of the hydrogel electrodes before use while in packaging, shipment, and storage and would be first to be removed during device use, Release liners 1002 and 1003 have cutouts corresponding to shape of the electrodes 107 108 while covering the adhesive layer 1005. These cutouts could be used to aid in the application of a liquid electrode gel without the liquid electrode gel contacting the adhesive layer 1005 while preventing unintended adhesion of the adhesive layer. Release liner 1002 has an overlapping portion over release liner 1003 and release liner 1003 has a protrusion outside the perimeter of adhesive layer 1005 to enhance the grip and removal of each liner.

### Methods of Use

Aspects of the present disclosure provide methods of treating or augmenting recovery from a medical condition of a subject, using the devices disclosed herein. An exemplary method may comprise the step of contacting the exemplary electrode assembly with the skin of the subject so that one or more of (a) the set of frontal electrodes one or more of overlie or straddle the supraorbital trigeminal nerve branch of the subject, or (b) the set of lateral electrodes one or more of overlie or straddle the auricular branch of the vagus nerve of the subject. The exemplary method may further comprise the step of transcutaneously delivering stimulation energy through one or more of the set of frontal electrodes or the set of lateral electrodes to treat the medical condition. The medical condition may be one or more of ischemic stroke, cerebral brain damage due to ischemic stroke, hemorrhagic stroke, cerebral brain damage due to hemorrhagic stroke, a reperfusion injury, traumatic brain injury, subarachnoid hemorrhage, a migraine, a headache, a form of dementia or cognitive impairment, a hematoma, a hemorrhage, a subarachnoid hemorrhage, inflammation, hypertension, hypotension, brain damage resulting from brain surgery, brain damage resulting from a brain resection, multiple sclerosis or lesions therefrom, cerebral palsy, or combinations thereof. In some embodiments, the method includes contacting the electrode assembly with the skin of the subject so that both (a) and (b) occur. In some embodiments, transcutaneously delivering stimulation energy comprises increasing a blood flow to the brain. In some embodiments, transcutaneously delivering stimulation energy comprises increasing a mean blood flow velocity to the brain, an increase in end diastolic velocity, a decrease in pulsatility index, or combinations thereof. In some embodiments, the superior frontal electrode has a triangular shape. In some embodiments, transcutaneously delivering stimulation energy comprises generating an electric field in an orientation of the supraorbital trigeminal nerve branch. In some embodiments, the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject. In some embodiments, transcutaneously delivering stimulation energy comprises reducing a current density applied to nerve fibers, and reducing the nerve fibers that are recruited upon generating a secondary phase of a pulse with a pulse generator. In some embodiments, transcutaneously delivering stimulation energy comprises reducing the area of an electric field produced by the superior frontal electrode relative to a semicircular shaped electrode. In some embodiments, transcutaneously delivering stimulation energy comprises increasing a stimulation effect to the subject at the same stimulation intensity relative to a stimulation effect resulting from a semicircular shaped electrode. In some embodiments, transcutaneously delivering stimulation energy comprises reducing off-target nerve activations when an electrical field is generated between the superior frontal electrode and the inferior frontal electrode and applied to the subject. In some embodiments, transcutaneously delivering stimulation energy comprises generating an electrical field in an orientation of an auricular branch of the vagus nerve with the set of lateral electrodes. In some embodiments, the orientation of the electrical field is parallel to one or more nerve fibers in the auricular branch of the vagus nerve. In some embodiments, transcutaneously delivering stimulation energy comprises minimizing off-target nerve activations when an electrical field is generated between the concha electrode and the tragus electrode and applied to the subject. In some embodiments, transcutaneously delivering stimulation energy comprises reducing off-target nerve activations when an electrical field is generated between the superior frontal electrode and the inferior frontal electrode. In some embodiments, the method includes attaching the electrode assembly to a head of the subject, wherein the electrode assembly is comprised in a head-worn apparatus for neural stimulation of target nerves in a subject, wherein the target nerve are the auricular branch of the vagus nerve and the supraorbital trigeminal nerve branch.

### EXAMPLES

The following illustrative examples are representative of embodiments of the devices and methods described herein and are not meant to be limiting in any way.

### Example 1: Superiority of Device Disclosed Herein Over Existing Devices for Treating Ischemic Stroke

In this example, subjects suffering from ischemic stroke and reperfusion injury are treated using electrodes from an existing commercially available device (e.g., the electrodes of the CEFALY® device) for trigeminal nerve stimulation in combination with an external pulse generator of the systems described herein; or using the devices, systems, electrodes, and methods described herein. The subjects present to the emergency department showing one or more symptoms of an ischemic stroke and are diagnosed with an ischemic stroke.

Shortly following diagnosis and in the pre-reperfusion therapy stage, a first subject is treated using an existing commercially available device electrodes for trigeminal nerve stimulation with the following stimulation parameters. Stimulating the first target nerve and the second target nerve comprises administering about a 100% trigeminal nerve stimulation duty cycle and about a 100% vagus nerve duty cycle constantly or continuously for treatment blocks between 1 minute to 4 hours, or until after reperfusion. Thereafter, stimulating the first target nerve and the second target nerve comprises administering about a 5-20% trigeminal nerve stimulation duty cycle and about a 10% vagus nerve duty cycle constantly or continuously, or every 4 hours; or another duty cycle. Mean flow velocity and pulsatility index are measured using transcranial Doppler ultrasound monitoring before, during, and after stimulation. Stimulating the target nerves as described mildly increases the flow of blood to the brain, mildly decreasing subject's pain.

A second subject is treated with the same stimulation parameters using an embodiment of the stimulation device described herein. The same stimulation parameters are applied as described above with the same external pulse generator. Mean flow velocity and pulsatility index are measured using transcranial Doppler ultrasound monitoring before, during, and after stimulation. Stimulating the target nerves as described herein increases the mean flow velocity by approximately 2% and decreases the pulsatility index by approximately 3% relative to the existing device during stimulation, with continued increased mean flow velocity and decreased pulsatility index after stimulation (see FIG. 7A, FIG. 7B), increasing the flow of blood to the brain, decreasing subject's pain, increasing the oxygenation level of penumbral tissue, and reducing the amount of penumbral tissue ultimately converted to an infarct core, and improving patient outcomes over the existing device. Mean cerebral blood flow velocity was increased using stimulation device described herein as compared to the existing commercially available device using similar or equivalent stimulation parameters as described above.

### Example 2: Superiority of Device Disclosed Herein over Earlier Prototype for Treating Ischemic Stroke

In this example, subjects suffering from ischemic stroke and reperfusion injury are treated using an earlier prototype which lacks features present in the tested device according to embodiments described herein, comprising alternate electric fields generated due to alternate electrode placement, or using the devices and methods described herein. The subjects present to the emergency department showing one or more symptoms of an ischemic stroke and are diagnosed with an ischemic stroke.

Shortly following diagnosis and in the pre-reperfusion therapy stage, a first subject is treated using an earlier prototype with the following stimulation parameters. Stimulating the first target nerve and the second target nerve comprises administering about a 100% trigeminal nerve stimulation duty cycle and about a 100% vagus nerve duty cycle constantly or continuously for treatment blocks between 1 minute to 4 hours, or until after reperfusion. Thereafter, stimulating the first target nerve and the second target nerve comprises administering about a 5-20% trigeminal nerve stimulation duty cycle and about a 10% vagus nerve duty cycle constantly or continuously, or every 4 hours; or another duty cycle. Mean flow velocity and pulsatility index are measured using transcranial Doppler ultrasound monitoring before, during, and after stimulation. Stimulating the target nerves as described mildly increases the flow of blood to the brain, mildly increases the oxygenation level of penumbral tissue, and mildly reduces the amount of penumbral tissue ultimately converted to an infarct core.

A second subject is treated with the same stimulation parameters using an embodiment of the stimulation device described herein, with the improved electrode design configuration, with the same stimulation parameters described above. Mean flow velocity and pulsatility index are measured using transcranial Doppler ultrasound monitoring before, during, and after stimulation. Stimulating the target nerves as described herein increases the mean flow velocity and decreases the pulsatility index during stimulation relative to the existing device, increasing the flow of blood to the brain, decreasing subject's pain, increasing the oxygenation level of penumbral tissue, and reducing the amount of penumbral tissue ultimately converted to an infarct core, and improving patient outcomes over the existing device with existing electrode placements.

Mean cerebral blood flow velocity was increased using the stimulation device described herein with the improved electrode design configuration as compared to the earlier prototype using similar or equivalent stimulation parameters as described above.

### Example 3: Testing Device Described Herein Against Existing Devices on a Healthy Subject

The devices and methods described herein were tested in comparison to a different electrode configuration using the electrodes of an existing commercially available trigeminal nerve stimulation device for the treatment of migraine (e.g., the electrodes of the CEFALY® device), while using the same external pulse generator. The CEFALY® device was chosen to demonstrate the difference between stimulation horizontally across the supraorbital branches and across a large non-targeted area of the forehead, as in the CEFALY® device, in comparison to stimulation vertically aligned with the supraorbital branches and across a smaller area targeted to the supraorbital notch or foramen, as is the case with the devices and methods described herein.

Testing was performed in a single healthy human subject (Male, 31 years old) over 6 stimulation sessions with at least 24 hours between each stimulation sessions. Stimulation sessions alternated between use of the devices and methods described herein and the CEFALY® electrodes with the same external pulse generator used with the devices described herein. The CEFALY® electrodes were placed centered on the subject's forehead just above the eyebrow to overlie the supraorbital nerve branches. The external pulse generator used for devices and methods described herein was applied to the CEFALY® device electrodes, instead of the CEFALY® device's accompanying stimulator.

During each stimulation session, stimulation was delivered for 1 minute every five minutes for a total of 3 stimulation periods. Stimulation parameters were the same for the CEFALY® device and the device described herein. Stimulation was applied to the two available poles on the Cephaly® electrodes. Stimulation was delivered using rectangular, biphasic, constant current pulses at 6 mA and 25 Hz. Stimulation was delivered as a 60 second pulse train of 25 Hz biphasic pulses. The first phase of the biphasic pulses was 6 mA and lasted 350 μs, followed by a 1 μs interphase period, followed by a second phase that was 2.4 mA and lasted for 875 μs. In the case of the CEFALY® device, pulses were delivered alternating between the left and right electrode as the leading cathodal pulse. In the case of the device described herein, pulses were delivered to both the left and right supraorbital locations and were non-alternating. Stimulation was not applied to the auricular branch. Current was not applied between the left and right sides. Current was only applied between the superior and inferior electrodes with the inferior electrode as the first phase cathode. The current of the pulses was linearly ramped up and down from 0 mA to 6 mA over the course of 5 seconds at the beginning and end of each stimulation period. The same stimulation parameters were applied to the device disclosed herein. Stimulation was not applied to the auricular branch.

During each stimulation session, transcranial Doppler (TCD) ultrasound was used to measure the flow velocity within the bilateral middle cerebral arteries (MCA) beginning 5 minutes before the initial stimulation period and continuing for 5 minutes after the final stimulation period. Pulsatility index (PI) was also calculated from the TCD ultrasound flow velocity waveform.

The measured mean flow velocity (MFV) and PI during each stimulation period were normalized to a pre-stimulation period baseline and averaged across stimulation periods and electrode designs. The results of this testing are depicted in FIG. 5, where the pre-stimulation baseline is shown during minutes 0 to 1, stimulation occurs during minute 1 to 2, and a post-stimulation period is shown in minutes 2 to 3.

While there was variability in the signal during the pre-stimulation period, upon stimulation with both electrodes MFV increased and PI decreased. Pulsatility index is a measure of vascular resistivity and is calculated from the difference between peak systolic velocity and end diastolic velocity divided by MFV. This pattern of increased MFV and decreased PI was indicative of distal perfusion increases within the microvasculature downstream of the MCA area measured. This pattern could be desirable to induce in acute ischemic stroke (AIS) patients or other neurological conditions while its opposite, increased PI and decreased MFV, has been shown to be associated with other neurological conditions such as Alzheimer's disease.

As shown in FIG. 6, under the same stimulation parameters, the devices and methods described herein produced a greater increase in MFV (e.g., about 2% to about 3% total increase) and a greater decrease (e.g., about 1% to about 4%) in PI during the stimulation period. After the cessation of stimulation, the CEFALY® device produced a larger increase in MFV (e.g., about 1% to about 2%) and more negative response in PI (e.g., about 1% to about 3%) than the devices and methods described herein, which more closely resembled the pre-stimulation baseline.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 4: Testing Device Described Herein in Subjects With Stroke Risk Factors

The devices and methods described herein were tested on subjects with risk factors for stroke in a clinical study. Testing was performed in a single session with ten participants (67.1±6.7 years of age, n=5 females, n=2 prior transient or cerebrovascular attack, n=7 hypertension, n=8hyperlipidemia, n=3 diabetes mellitus). Stimulation was performed using the external pulse generator used for devices and methods described herein. Transcranial doppler ultrasonography was used to record the cerebral blood flow velocity M1 segment of the middle cerebral artery and compare blood flow metrics before and during stimulation

During each stimulation session, stimulation was initially delivered in a tolerability assessment period. Stimulation was delivered using rectangular, biphasic, constant current pulses at up to 12 mA at the frontal supraorbital branch electrode locations and up to 5 mA at the lateral auricular branch electrode locations. The amplitude of the biphasic pulses was gradually increased linearly at all locations simultaneously from a value of 0 mA to a submaximal level of tolerability of the participant and was delivered as 25 Hz biphasic pulses. Shortly after the tolerability assessment period, a period of intermittent stimulation was delivered in 3-minute pulse trains beginning every 6 minutes. The intermittent stimulation period was conducted with a duration of at least 40 minutes per participant. During each 3-minute pulse train, the intensity level of the stimulation pulses was delivered according to a pre-determined algorithm that varied the intensity level below the highest level of the tolerability assessment period. Similarly, during each 3-minute pulse train, the frequency of the stimulation pulses was delivered according to a pre-determined algorithm that varied the pulse frequency around the setting of the tolerability assessment period.

During each stimulation session, transcranial Doppler (TCD) ultrasound was used to measure the flow velocity within the M1 segment of the bilateral middle cerebral arteries (MCA) beginning 10 minutes before the initial stimulation period and continuing for 5 minutes after the final stimulation period.

The measured mean flow velocity (MFV) and end diastolic velocity (EDV) during each stimulation period were normalized to a pre-stimulation period baseline. and averaged across stimulation periods and pulse amplitude (intensity level). An example of results from testing during the tolerability assessment period on MFV are depicted in FIG. 8A and on EDV are depicted in FIG. 8B. A line of best fit for all participants is shown on FIG. 8C and FIG. 8D. During tolerability assessment, a 10% increase in stimulation intensity predicted a 1.7% increase in mean flow velocity compared to initial baseline, β=0.017, R²=0.29, F(2,210)=86.3, p<0.001, and a 1.6% increase in end diastolic velocity (EDV) compared to initial baseline, β=0.016, R²=0.22, F(2,210)=58.5, p<0.001. In addition, during each 3-minute pulse train of the intermittent stimulation period, stimulation was associated with a 17% reduction in min-PI relative to the prior inter-stimulus interval, β=−0.173, 95% CI [−0.273, −0.073], p<0.001. During this clinical testing, no severe adverse events were reported and participants graded comfort as [] on a scale of 10, with 10 being the most comfortable.

Upon stimulation with an exemplary stimulation device, both MFV increased and EDV increased immediately in response to stimulation, while PI decreased during the prolonged intermittent stimulation periods. Combined, these measures are indicative of cerebral vasodilation. These results could be clinically meaningful. For example, in acute ischemic stroke patients with proximal intracranial occlusion that are treated with a thrombolytic, an increase in end diastolic velocity has been shown to be independently associated with declines in the National Institutes of Health Stroke Scale score, early neurological improvement, and favorable functional outcomes. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An electrode assembly for neural stimulation of a plurality of target nerves in a subject, the assembly comprising:
i) a substrate comprising a plurality of electrical connections;
ii) a first set of electrodes, comprising one or more of:
(a) a set of frontal electrodes comprising a superior frontal electrode and an inferior frontal electrode, the set of frontal electrodes positioned on the substrate such that the set of frontal electrodes are configured to contact the subject's skin and to one or more of overlie or straddle at least a first portion of a supraorbital trigeminal nerve branch of the subject; or
(b) a set of lateral electrodes comprising a concha electrode and a tragus electrode, the set of lateral electrodes positioned on the substrate such that the set of lateral electrodes are configured to contact the subject's skin and to one or more of overlie or straddle at least a first portion of an auricular branch of the vagus nerve of the subject;
wherein the substrate includes a fixation promoter configured to promote contact with each electrode and the subject's skin, or promote contact between the substrate and the subject;
wherein each electrode in the first set of electrodes is in communication with at least one of the plurality of electrical connections.

2. The electrode assembly of claim 1, further comprising a second set of frontal electrodes comprising a second superior frontal electrode and a second inferior frontal electrode, wherein the second set of frontal electrodes are positioned on the substrate such that the second set of frontal electrodes are configured to contact the subject's skin and to one or more of overlie or straddle at least a second portion of the supraorbital trigeminal nerve branch of the subject.

3. (canceled)

4. The electrode assembly of claim 1, further comprising an adhesive layer surrounding each electrode, wherein the adhesive layer adheres the electrodes to the skin of the subject.

5. (canceled)

6. The electrode assembly of claim 1, wherein at least one of the electrodes comprises a hydrogel enhancing current transmission between a pulse generator and a respective target nerve, wherein the target nerve is the auricular branch of the vagus nerve or the supraorbital trigeminal nerve branch.

7. The electrode assembly of claim 1, wherein a pulse generator transmit current from the superior frontal electrode to the inferior frontal electrode.

8. The electrode assembly of claim 1, wherein the inferior frontal electrode is shaped and/or positioned on the substrate for placement over a nerve bundle above the supraorbital foramen, the supraorbital notch, or both.

9. The electrode assembly of claim 1, wherein the first set of electrodes is configured to generate an electric field in an orientation of the target nerves branch.

10. The electrode assembly of claim 1, wherein the superior frontal electrode has a triangular shape and generates an electric field in an orientation of the supraorbital trigeminal nerve branch.

11. The electrode assembly of claim 10, wherein the orientation of the electrical field is parallel to one or more nerve fibers in the supraorbital trigeminal nerve branch, or wherein a gradient of the electrical field changes in a same direction as a trajectory of the supraorbital branch of the trigeminal nerve on a forehead of the subject.

12. The electrode assembly of claim 10, wherein the triangular shape of the superior frontal electrode reduces a current density applied to nerve fibers, reducing the nerve fibers that are recruited on a secondary phase of a pulse of a pulse generator.

13. The electrode assembly of claim 1, wherein the superior frontal electrode has a triangular shape and reduces the area of an electric field produced by the electrode.

14.-21. (canceled)

22. The electrode assembly of claim 1, further comprising a mechanical tensioner substantially along at least a portion of a length of the electrode assembly applying a compressive pressure to maintain contact between the electrode assembly and the skin of a user.

23. (canceled)

24. The electrode assembly of claim 1, wherein the fixation promoter comprises at least one of an elastic headband and/or a frame.

25.-28. (canceled)

29. The electrode assembly of claim 1, wherein the set of lateral electrodes are removable from the device.

30.-31. (canceled)

32. The electrode assembly of claim 1, wherein the electrode assembly is comprised in a head-worn apparatus for neural stimulation of target nerves in a subject, wherein the target nerve are the auricular branch of the vagus nerve and the supraorbital trigeminal nerve branch.

33.-75. (canceled)

76. The electrode assembly of claim 1, wherein the subject has a medical condition comprising one or more of ischemic stroke, cerebral brain damage due to ischemic stroke, hemorrhagic stroke, cerebral brain damage due to hemorrhagic stroke, a reperfusion injury, traumatic brain injury, subarachnoid hemorrhage, a migraine, a headache, a form of dementia or cognitive impairment, a hematoma, a hemorrhage, a subarachnoid hemorrhage, inflammation, hypertension, hypotension, brain damage resulting from brain surgery, brain damage resulting from a brain resection, multiple sclerosis or lesions therefrom, cerebral palsy, or combinations thereof.

77. The electrode assembly of claim 1, wherein the first set of electrodes is configured for neural stimulation via one or more of electrical, mechanical, vibratory, acoustic, optical, or thermal energy.

78. The electrode assembly of claim 1, wherein the set of frontal electrodes is configured to overlie a forehead of the subject.

79. The electrode assembly of claim 1, wherein the substrate comprises a forehead section having a shape and area for placement over at least a portion of a supraorbital trigeminal nerve branch of the subject.

80. The electrode assembly of claim 22, wherein the mechanical tensioner comprises at least one of a strap or a curved material.
